(19) United States
(12) Patent Application Publication
(10) Pub. No.: US 2026/0026860 A1
(43) Pub. Date: Jan. 29, 2026

(54) ELECTROSURGICAL INSTRUMENT WITH IMPEDANCE SPECTROSCOPY AND METHOD OF MONITORING STATE OF INSTRUMENT JAWS AND TISSUE

(71) Applicant: Cilag GmbH International, Zug (CH)

(72) Inventors: Madison K. Vanosdoll, Cincinnati, OH (US); Geoffrey S. Strobl, Williamsburg, OH (US); Raymond E. Parfett, Cincinnati, OH (US); Chad P. Boudreaux, Cincinnati, OH (US); Steven M. Boronyak, Cincinnati, OH (US); Meredith N. Lusk, Dearbon Heights, MI (US); Eric B. LaFay, Madeira, OH (US); Narendran Narasimhan, Cincinnati, OH (US); David J. Maher, Philadelphia, PA (US)

(21) Appl. No.: 18/783,643

(22) Filed: Jul. 25, 2024

(51) Int. Cl. A61B 18/14; A61B 18/00; A61B 18/12; A61B 90/00

(52) U.S. Cl. CPC A61B 18/1445; A61B 18/1206; A61B 90/06; A61B 2018/00184; A61B 2018/00601; A61B 2018/0063; A61B 2018/00642; A61B 2018/00875; A61B 2018/1455; A61B 2090/061

(57) ABSTRACT

A surgical instrument includes an end effector and a controller. The end effector includes a first jaw and a second jaw that can grasp tissue such that the end effector may apply therapeutic energy to seal the grasped tissue. The end effector further includes a knife that can sever the grasped tissue, and a electrode that can engage the grasped tissue and emit non-therapeutic energy. The controller can measure and analyze an impedance phase angle and an impedance magnitude in response to the emitted non-therapeutic energy. Further, the controller can detect a notable event at least partially based on the impedance phase angle or the impedance magnitude.

### BACKGROUND

A variety of surgical instruments include a tissue cutting element and one or more elements that transmit radio frequency (RF) energy to tissue (e.g., to coagulate or seal the tissue). An example of such an electrosurgical instrument is the ENSEAL® Tissue Sealing Device by Ethicon Endo-Surgery, Inc., of Cincinnati, Ohio. In some instances, it may be desirable to monitor the state of an end effector of such an electrosurgical instrument during illustrative use.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary electrosurgical instrument;

FIG. 2 depicts a perspective view of an exemplary articulation assembly and end effector of the electrosurgical instrument of FIG. 1;

FIG. 3 depicts an exploded view of the articulation assembly and end effector of FIG. 2;

FIG. 4 depicts a perspective view of the end effector that of FIG. 2;

FIG. 5 depicts an exploded perspective view of the end effector of FIG. 2;

FIG. 6 depicts an illustrative impedance triangle;

FIG. 7 depicts a set of illustrative example waveforms;

FIG. 8 depicts another set of illustrative example waveforms;

FIG. 9 depicts another illustrative example waveform;

FIG. 10 depicts another illustrative example waveform;

FIG. 11 depicts another set of illustrative example waveforms;

FIG. 12 depicts another illustrative example waveform;

FIG. 13 depicts another illustrative example waveform;

FIG. 14 depicts a graph of an illustrative example of impedance magnitudes measured over time utilizing non-therapeutic impedance sensing during illustrative use of the electrosurgical instrument of FIG. 1;

FIG. 15 depicts graph of an illustrative example of impedance phase angles measured over time utilizing non-therapeutic impedance sensing during illustrative use of the electrosurgical instrument of FIG. 1;

FIG. 16A depicts a cross-sectional view of the end effector of FIG. 2, with a pair of jaws of the end effector in an open position with tissue therebetween;

FIG. 16B depicts a cross-sectional view of the end effector of FIG. 2, with the pair of jaws of FIG. 16A being initially closed toward the tissue therebetween;

FIG. 16C depicts a cross-sectional view of the end effector of FIG. 2, with the pair of jaws of FIG. 16A in the clamped position, thereby clamping tissue therebetween;

FIG. 16D depicts a cross-sectional view of the end effector of FIG. 2, with the pair of jaws of FIG. 16A in the clamped position, thereby clamping tissue therebetween, wherein the pair of jaws are activated with therapeutic energy to seal the clamped tissue;

FIG. 16E depicts a cross-sectional view of the end effector of FIG. 2, with the pair of jaws of FIG. 16A in the clamped position, thereby clamping tissue therebetween, wherein a knife is fired to seal the clamped tissue;

FIG. 16F depicts a cross-sectional view of the end effector of FIG. 2, with the pair of jaws of FIG. 16A in the clamped position, thereby clamping tissue therebetween, wherein the knife of FIG. 16E is retracted;

FIG. 16G depicts a cross-sectional view of the end effector of FIG. 2, with the pair of jaws of FIG. 16A in the open position, thereby releasing recently sealed and severed tissue;

FIG. 17 depicts a flowchart of an illustrative method of use of the end effector of FIG. 2;

FIG. 18 depicts a flowchart of another illustrative method of use of the end effector of FIG. 2;

FIG. 19 depicts a graph of an illustrative example of impedance magnitudes measured over time utilizing non-therapeutic impedance sensing during illustrative clamping of the electrosurgical instrument of FIG. 1;

FIG. 20 depicts a graph of an illustrative example of impedance phase angles measured over time utilizing non-therapeutic impedance sensing during illustrative clamping of the electrosurgical instrument of FIG. 1;

FIG. 21 depicts a flowchart of an illustrative method of use of the end effector of FIG. 2;

FIG. 22 depicts a perspective view of an alternative end effector;

FIG. 23 depicts a flowchart of an illustrative method of use of the end effector of FIG. 22;

FIG. 24 depicts a perspective view of an alternative electrosurgical instrument;

FIG. 25 depicts a flowchart of an illustrative method of use of the instrument of FIG. 24; and

FIG. 26 depicts a perspective view of an alternative handle for an electrosurgical instrument.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

### I. EXAMPLE OF ELECTROSURGICAL INSTRUMENT

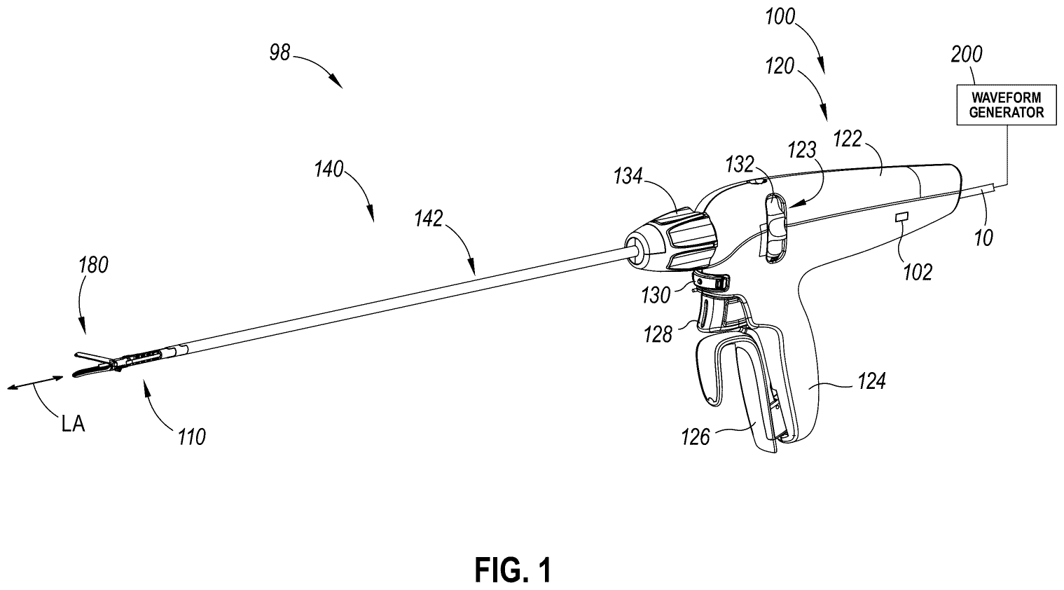

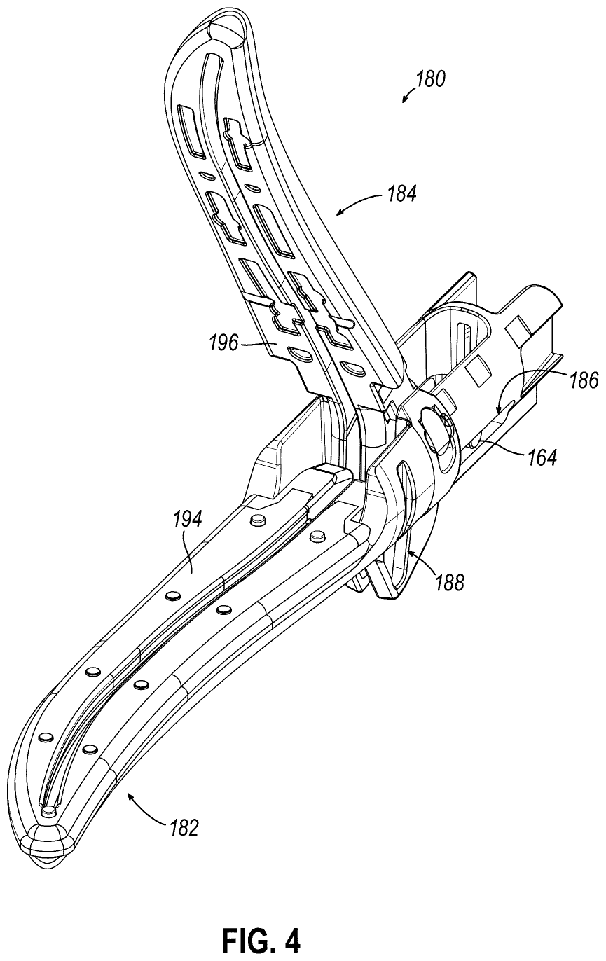

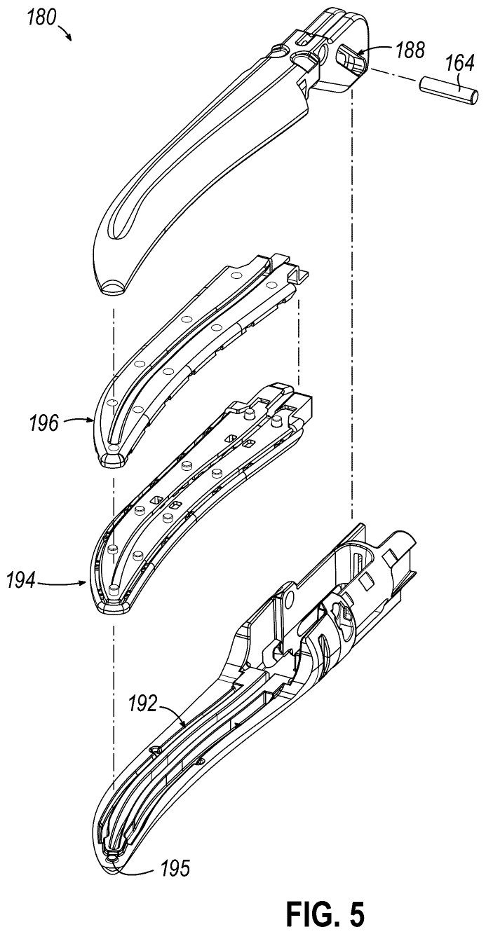

FIGS. 1-5 show a surgical system (98) including an exemplary electrosurgical instrument (100). As best seen in FIG. 1, electrosurgical instrument (100) includes a handle assembly (120), a shaft assembly (140), an articulation assembly (110), which may also be referred to as an articulation section (110), and an end effector (180). As will be described in greater detail below, end effector (180) of electrosurgical instrument (100) is operable to grasp, cut, and seal or weld tissue (e.g., a blood vessel, etc.). In this example, end effector (180) is configured to apply a non-therapeutic bipolar radio frequency (RF) energy in order to identify and/or verify that the correct tissue is present in the end effector such that a therapeutic RF energy can be applied to seal or weld tissue. However, it should be understood that electrosurgical instrument (100) may be configured to seal or weld tissue through any other suitable means that would be apparent to one skilled in the art in view of the teachings herein. For example, electrosurgical instrument (100) may be configured to seal or weld tissue via an ultrasonic blade, staples, etc. In the present example, electrosurgical instrument (100) is electrically coupled to a waveform generator (200) of surgical system (98), which is capable of delivering therapeutic and non-therapeutic energy, via power cable (10).

Waveform generator (200) may be configured to provide all or some of the electrical power requirements for use of electrosurgical instrument (100). Any suitable waveform generator (200) may be used as would be apparent to one skilled in the art in view of the teachings herein. By way of non-limiting example, the waveform generator (200) may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein, in its entirety. While in the current example, electrosurgical instrument (100) is coupled to waveform generator (200) via power cable (10), electrosurgical instrument (100) may contain an internal power source or plurality of power sources, such as a battery and/or supercapacitors, to electrically power electrosurgical instrument (100). Of course, any suitable combination of power sources may be utilized to power electrosurgical instrument (100) as would be apparent to one skilled in the art in view of the teaching herein.

Handle assembly (120) is configured to be grasped by an operator with one hand, such that an operator may control and manipulate electrosurgical instrument (100) with a single hand. Although electrosurgical instrument (100) is primarily described herein as being used by a human user, it should be noted that alternative versions exist in which one or more robotic systems (e.g., a robotic arm) may be used to control and manipulate electrosurgical instrument (100). Shaft assembly (140) extends distally from handle assembly (120) and connects to articulation assembly (110). Articulation assembly (110) is also connected to a proximal end of end effector (180). As will be described in greater detail below, components of handle assembly (120) are configured to control end effector (180) such that an operator may grasp, cut, and seal or weld tissue. Articulation assembly (110) is configured to deflect end effector (180) from the longitudinal axis (LA) defined by shaft assembly (140).

Handle assembly (120) of the present example includes a control unit (102) housed within a body (122), a pistol grip (124), a jaw closure trigger (126), a knife trigger (128), an activation button (130), an articulation control (132), and a knob (134). As will be described in greater detail below, jaw closure trigger (126) may be pivoted toward and away from pistol grip (124) and/or body (122) to open and close jaws (182, 184) of end effector (180) to grasp tissue. Additionally, knife trigger (128) may be pivoted toward and away from pistol grip (124) and/or body (122) to actuate a knife member (176) within the confines of jaws (182, 184) to cut tissue captured between jaws (182, 184). Further, activation button (130) may be pressed to apply radio frequency (RF) energy to tissue via electrodes (194, 196) of jaws (182, 184), respectively. In some versions, electrodes (194, 196) of jaws (182, 184) are in a bifurcation configuration where electrodes (194, 196) move relative to a central axis and nearly equal and opposite to one another.

Body (122) of handle assembly (120) defines an opening (123) through which a portion of articulation control (132) protrudes. Articulation control (132) is rotatably disposed within body (122) such that an operator may rotate the portion of articulation control (132) protruding from opening (123) to rotate the portion of articulation control (132) located within body (122). Rotation of articulation control (132) relative to body (122) will bend articulation assembly (110) in order to drive deflection of end effector (180) from the longitudinal axis (LA) defined by shaft assembly (140). Articulation control (132) and articulation assembly (110) may include any suitable features to drive deflection of end effector (180) from the longitudinal axis (LA) defined by shaft assembly (140) as would be apparent to one skilled in the art in view of the teachings herein.

Knob (134) is rotatably disposed on the distal end of body (122) and is configured to rotate end effector (180), articulation assembly (110), and shaft assembly (140) about the longitudinal axis (LA) of shaft assembly (140) relative to handle assembly (120). While in the current example, end effector (180), articulation assembly (110), and shaft assembly (140) are rotated by knob (134), knob (134) may be configured to rotate end effector (180) and articulation assembly (110) relative to selected portions of shaft assembly (140). Knob (134) may include any suitable features to rotate end effector (180), articulation assembly (110), and shaft assembly (140) as would be apparent to one skilled in the art in view of the teachings herein.

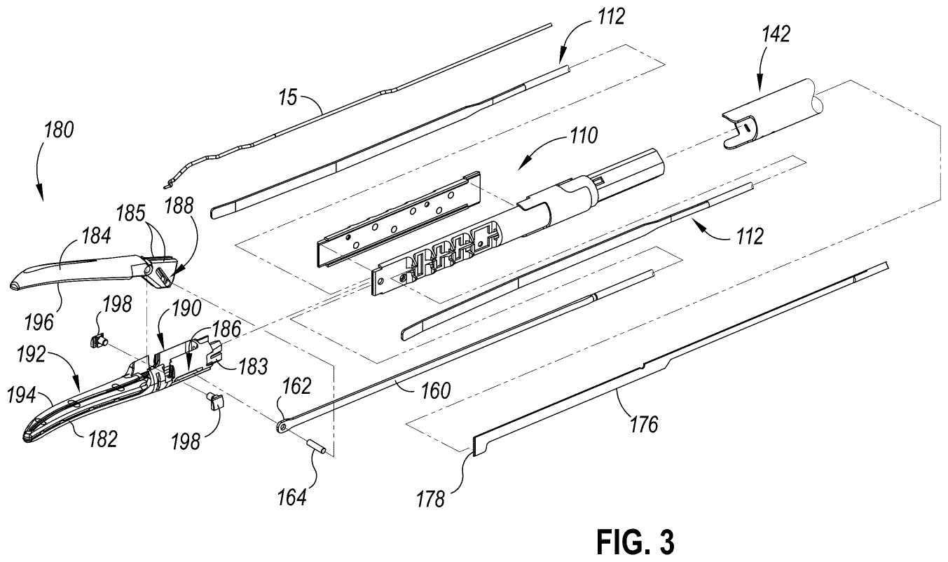

Shaft assembly (140) includes distal portion (142) extending distally from handle assembly (120) and a proximal portion housed within the confines of body (122) of handle assembly (120). Referring to FIG. 3, shaft assembly (140) houses a jaw closure connector (160) that couples jaw closure trigger (126) with end effector (180). Additionally, shaft assembly (140) houses a portion of knife member (176) extending between distal a distal cutting edge (178) of knife member (176) and knife trigger (128). Shaft assembly (140) also houses actuating members (112) that couple articulation assembly (110) with articulation control (132); as well as an electrical coupling (15) that operatively couples electrodes (194, 196) with activation button (130). As will be described in greater detail below, jaw closure connector (160) is configured to translate relative to shaft assembly (140) to open and close jaws (182, 184) of end effector (180); while knife member (176) is coupled to knife trigger (128) of handle assembly (120) to translate distal cutting edge (178) within the confines of end effector (180); and activation button (130) is configured to activate electrodes (194, 196).

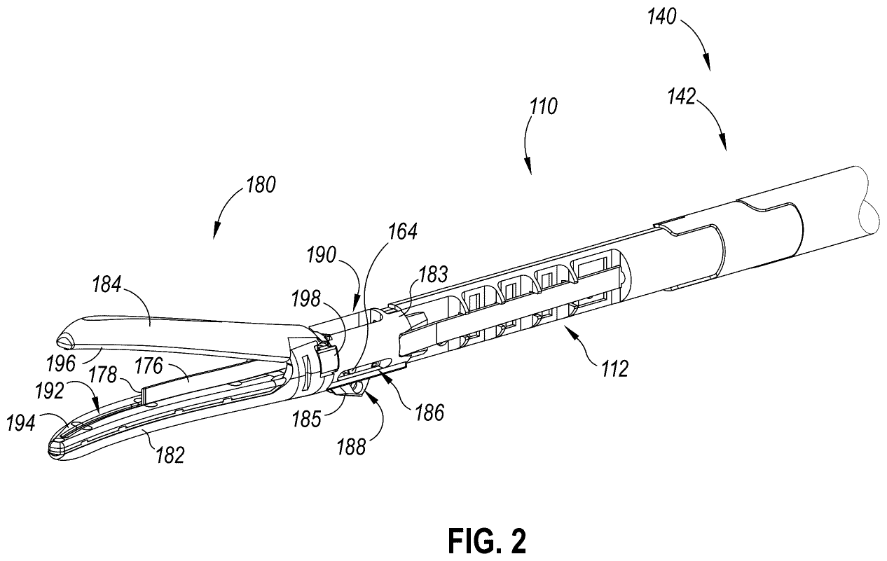

As best seen in FIGS. 2-5, end effector (180) includes lower jaw (182) pivotally coupled with upper jaw (184) via pivot couplings (198). Lower jaw (182) includes a proximal body (183) defining a slot (186), while upper jaw (184) includes proximal arms (185) defining a slot (188). Lower jaw (182) also defines a central channel (190) that is configured to receive proximal arms (185) of upper jaw (184), portions of knife member (176), jaw closure connector (160), and pin (164). Slots (186, 188) each slidably receive pin (164), which is attached to a distal coupling portion (162) of jaw closure connector (160). Additionally, lower jaw (182) includes a force sensor (195) located at a distal tip of lower jaw (182), though force sensor (195) may alternatively be positioned at any other suitable location. Force sensor (195) may be in communication with control unit (102). Force sensor (195) may be configured to measure the closure force generated by pivoting jaws (182, 184) into a closed configuration in accordance with the description herein. Additionally, force sensor (195) may communicate this data to control unit (102). Any suitable components may be used for force sensor (195) as would be apparent to one skilled in art in view of the teachings herein. For example, force sensor (195) may take the form of a strain gauge. In some variations, end effector (180) includes more than one force sensor.

While in the current example, a force sensor (195) is incorporated into electrosurgical instrument (100) and is in communication with control unit (102), any other suitable sensors or feedback mechanisms may be additionally or alternatively incorporated into electrosurgical instrument (100) while in communication with control unit (102) as would be apparent to one skilled in the art in view of the teachings herein. For instance, an articulation sensor or feedback mechanism may be incorporated into electrosurgical instrument (100), where the articulation sensor communicates signals to control unit (102) indicative of the degree end effector 180 is deflected from the longitudinal axis (LA) by articulation control (132) and articulation assembly (110).

As will be described in greater detail below, jaw closure connector (160) is operable to translate within central channel (190) of lower jaw (182). Translation of jaw closure connector (160) drives pin (164). As will also be described in greater detail below, with pin (164) being located within both slots (186, 188), and with slots (186, 188) being angled relative to each other, pin (164) cams against proximal arms (185) to pivot upper jaw (184) toward and away from lower jaw (182) about pivot couplings (198). Therefore, upper jaw (184) is configured to pivot toward and away from lower jaw (182) about pivot couplings (198) to grasp tissue.

The term “pivot” does not necessarily require rotation about a fixed axis and may include rotation about an axis that moves relative to end effector (180). Therefore, the axis at which upper jaw (184) pivots about lower jaw (182) may translate relative to both upper jaw (184) and lower jaw (182). Any suitable translation of the pivot axis may be used as would be apparent to one skilled in the art in view of the teachings herein.

Lower jaw (182) and upper jaw (184) also define a knife pathway (192). Knife pathway (192) is configured to slidably receive knife member (176), such that knife member (176) may be retracted, and advanced, to cut tissue captured between jaws (182, 184).

Lower jaw (182) and upper jaw (184) each comprise a respective electrodes (194, 196). The power source may provide RF energy to electrodes (194, 196) via electrical coupling (15) that extends through handle assembly (120), shaft assembly (140), articulation assembly (110), and electrically couples with one or both of electrodes (194, 196). Electrical coupling (15) may selectively activate electrodes (194, 196) in response to an operator pressing activation button (130). In some instances, control unit (102) may couple electrical coupling (15) with activation button (130), such that control unit (102) activates electrodes (194, 196) in response to operator pressing activation button (130). Control unit (102) may have any suitable components in order to perform suitable functions as would be apparent to one skilled in the art in view of the teachings herein. For instance, control unit (102) may have a processor, memory unit, suitable circuitry, etc. Examples of features and functionalities that may be incorporated into control unit (102) will be described in greater detail below.

As described above, jaw closure trigger (126) may be pivoted toward and away from pistol grip (124) and/or body (122) to open and close jaws (182, 184) of end effector (180) to grasp tissue. In particular, as will be described in greater detail below, pivoting jaw closure trigger (126) toward pistol grip (124) may proximally actuate jaw closure connector (160) and pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

In some versions, knife trigger (128) may be pivoted toward and away from body (122) and/or pistol grip (124) to actuate knife member (176) within knife pathway (192) of jaws (182, 184) to cut tissue captured between jaws (182, 184). In particular, handle assembly (120) further includes a knife coupling body that is slidably coupled along proximal portion of shaft assembly (140). Knife coupling body is coupled with knife member (176) such that translation of knife coupling body relative to proximal portion of shaft assembly (140) translates knife member (176) relative to shaft assembly (140).

In another version, knife coupling body may be coupled to a knife actuation assembly such that as knife trigger (128) pivots toward body (122) and/or pistol grip (124), knife actuation assembly drives knife coupling body distally, thereby driving knife member (176) distally within knife pathway (192). Because knife coupling body is coupled to knife member (176), knife member (176) translates distally within shaft assembly (140), articulation assembly (110), and within knife pathway (192) of end effector (180). Knife member (176) includes distal cutting edge (178) that is configured to sever tissue captured between jaws (182, 184). Therefore, pivoting knife trigger (128) causes knife member (176) to actuate within knife pathway (192) of end effector (180) to sever tissue captured between jaws (182, 184).

With distal cutting edge (178) of knife member (176) actuated to the advanced position, an operator may press activation button (130) to selectively activate electrodes (194, 196) of jaws (182, 184) to seal or weld severed tissue captured between jaws (182, 184). It should be understood that the operator may also press activation button (130) to selectively activate electrodes (194, 196) of jaws (182, 184) at any suitable time during exemplary use. Therefore, the operator may also press activation button (130) while knife member (176) is retracted. Next, the operator may release jaw closure trigger (126) such that jaws (182, 184) pivot into the opened configuration, releasing tissue.

### II. SENSING TISSUE IMPEDANCE FOR DETERMINATIONS OF TISSUE STATE

Electrosurgical instrument (100) discussed above is configured to clamp tissue using end effector (180). Once securely clamped, electrodes (194, 196) in end effector (180) apply a non-therapeutic (i.e., low voltage) waveform to the tissue; and sensor devices measure the returning waveform to calculate and measure the impedance of the tissue. In one example, one or more electrodes (194, 196) are operatively connected to such sensor devices such that electrodes (194, 196) may be referred to as sensors in this respect. More specifically, electrosurgical instrument (100), via one or more sub-circuits, will provide non-therapeutic energy to the extracellular and intracellular fluid present within a given (e.g., clamped) region of tissue to determine a phase and a magnitude of the impedance of the tissue within jaws (182, 184). A processor may then relay information associated with a state of the tissue, such as, for example, tissue type, tissue phase, tissue margin, and the like. Using this associated information, a system inclusive of such electrosurgical instrument (100) cannot only verify that the proper tissue is clamped between jaws (182, 184), but can also determine if any non-tissue material is present between jaws (182, 184), and/or if a proper seal has been created after applying the therapeutic RF energy.

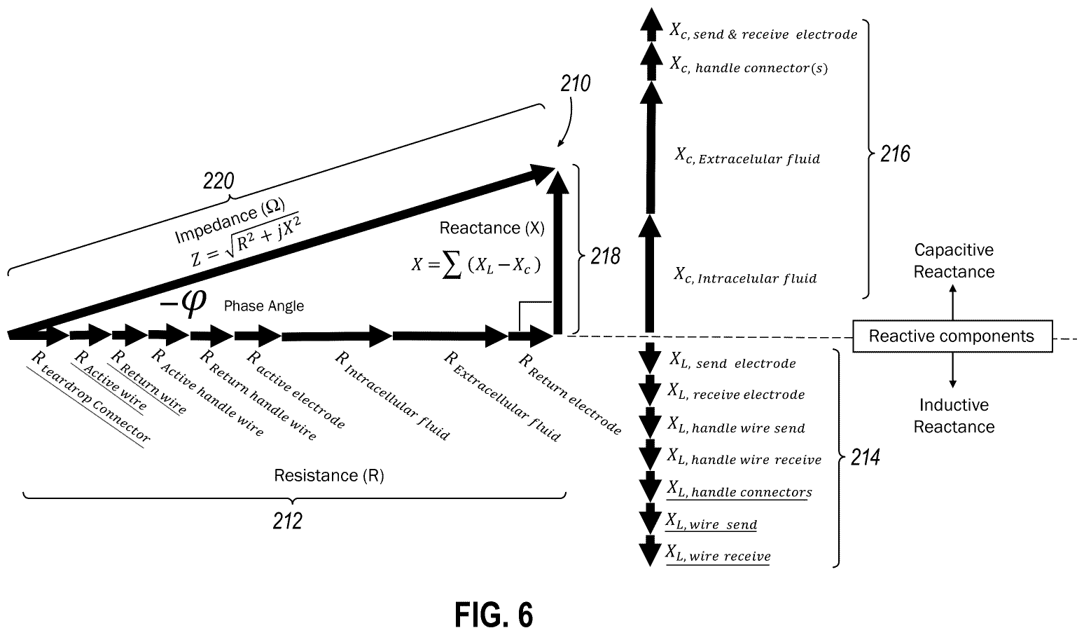

FIG. 6 shows an illustrative impedance triangle (210). As would be understood by one skilled in the art, human tissues may tend to be capacitive in nature, while wires, tool, staples, implants, etc. may tend to be inductive in nature. Thus, as can be seen by the illustrative impedance triangle (210), the “resistance” of each object in the circuit is measured (212) using the waveform and sensor electrodes (194, 196). Such system can also determine the “capacitive reactance” of each object in the circuit and the inductive reactance of each object in the circuit. For example, the send and receive electrodes (194, 196), the send and receive handle wires, the handle connector and the send and receive wires (e.g., included in power cable 10 (see FIG. 1)) all have inductive reactance (214). Additionally, the send and receive electrodes (194, 196), the handle connector, the extracellular fluid, and the intracellular fluid all have capacitive reactance (216). The “reactance” (218) can then be calculated by determining the difference between the capacitive reactance and the inductive reactance using:

$$X = \sum ( {X}_{L} - {X}_{C} ) . \qquad Equation 1$$

As shown in FIG. 6, the “impedance” 1406 can then be determined using:

$$Z = \sqrt{{R}^{2} + {jX}^{2}} \qquad Equation 2$$

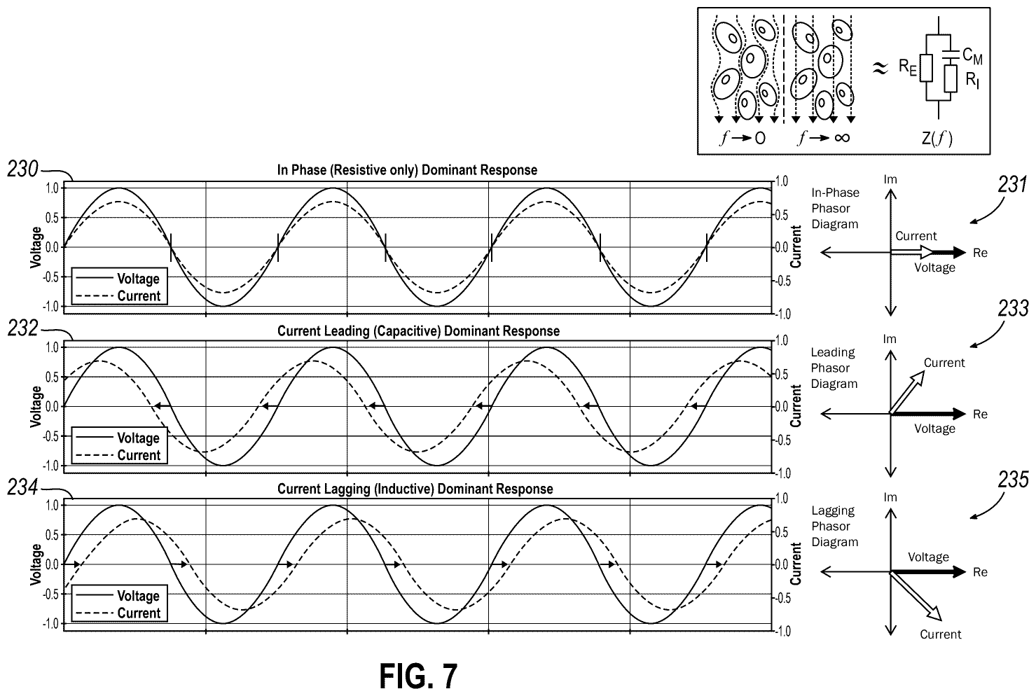

FIG. 7 shows a set of illustrative example waveforms. As would be understood by one skilled in the art, if a circuit only contains resistive items, the current and voltage will remain in phase such as shown in a first graph (230) and a first phasor diagram (231). Alternatively, if the circuit has capacitive objects, or more capacitive than inductive, the voltage wave will lead the current wave such as shown in a second graph (232) and a second phasor diagram (233). Finally, if the circuit has inductive objects, or more inductive objects than capacitive objects, the voltage will lag behind the current, such as shown in a third graph (234) and third phasor diagram (235).

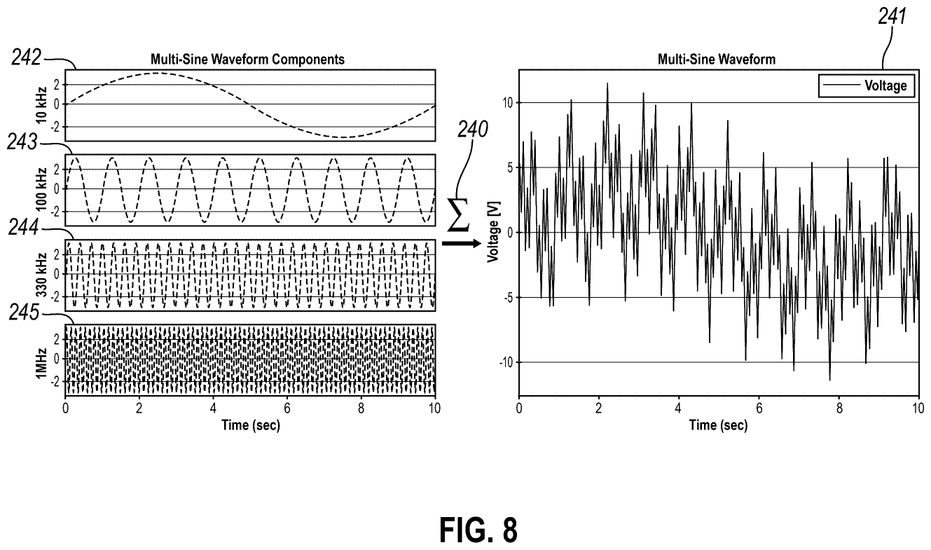

As discussed herein, the system may pass a non-therapeutic waveform through a portion of patient tissue to help identify the type of tissue as well as any foreign objects. Thus, in some versions, the system may pass waveforms of varying frequency (e.g., in series and/or parallel) to improve the accuracy of the determination. Accordingly, in some versions, and as shown in FIG. 8, multiple waveforms of various frequencies may be added or summed together (240) to create a muti-sine waveform (241). By way of non-limiting example, a 10 kHz sine wave (242) may be combined with a 100 kHz sine wave (243), a 330 kHz sine wave (244) and a 1 MHz sine wave (245) may be combined to create multi-sine wave (241).

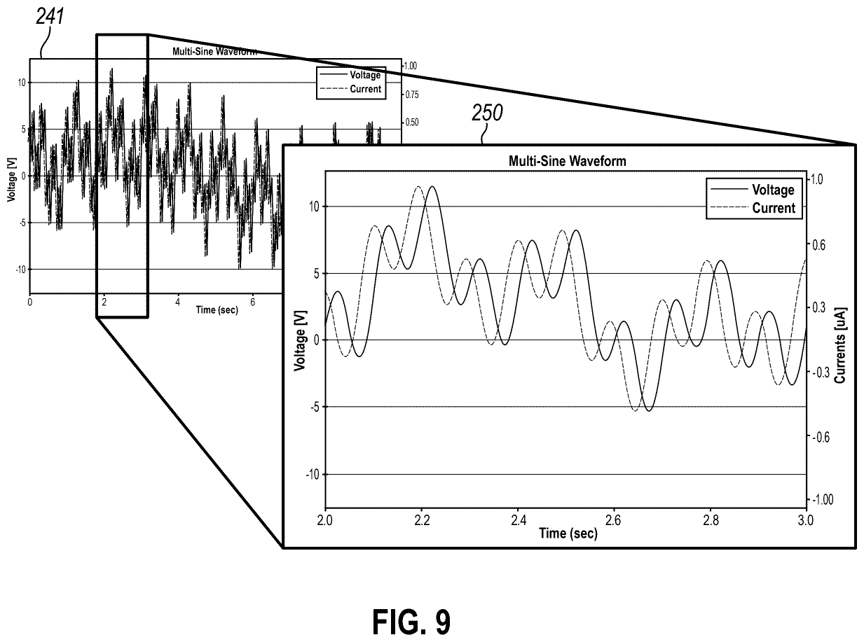

Referring now to FIG. 9, multi-sine waveform (241) may be sampled or windowed. In some versions, such as those that require the use of Fast Fourier Transforms (FFT), the windowing or sampling may be as small as a single period for the lower frequency waveform. As shown in a fourth graph (250), the voltage of multi-sine waveform (241) is leading the current and thus in the present version indicates a capacitive circuit (e.g., likely tissue). In an alternative version, the system may apply a series of burst waveforms having different frequencies.

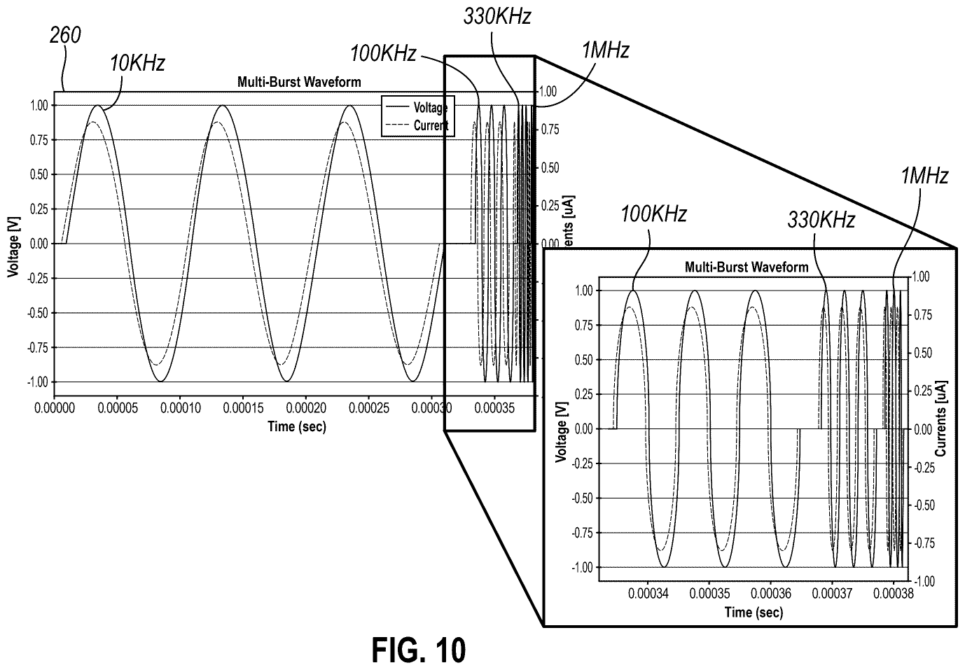

Referring now to FIG. 10, a burst waveform, including a brief delay between frequencies, is shown in fifth graph (260). In some versions, and as shown, the system may output a burst waveform that is a sine wave, while in other versions, the wave may be a square, triangle, ramp, pulse, pseudorandom binary sequence (PRBS), or arbitrary waveform. In some versions, the pause between waveforms can be evaluated in order to determine a “rebounding” time. The rebounding time may be used to help identify tissue types by evaluating how long certain tissues take to allow the waveform and any residual energy to dissipate from the tissue.

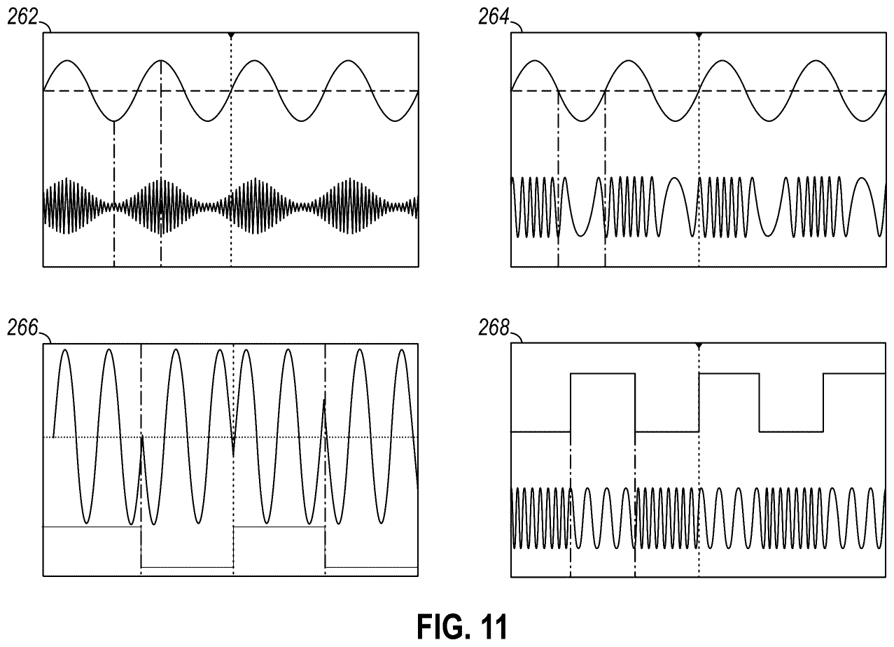

FIG. 11 shows various alternative burst versions. Specifically, in one version, amplitude modulation (AM) (262) may be used; while in another version, frequency modulation (FM) (264). Other versions may use phase modulation (PM) (266) and/or frequency-shift keying (FSK) modulation (268). Due to the fact that all of the modulation options shown in FIG. 11 involve a shift of some type, they may all be evaluated in a similar manner.

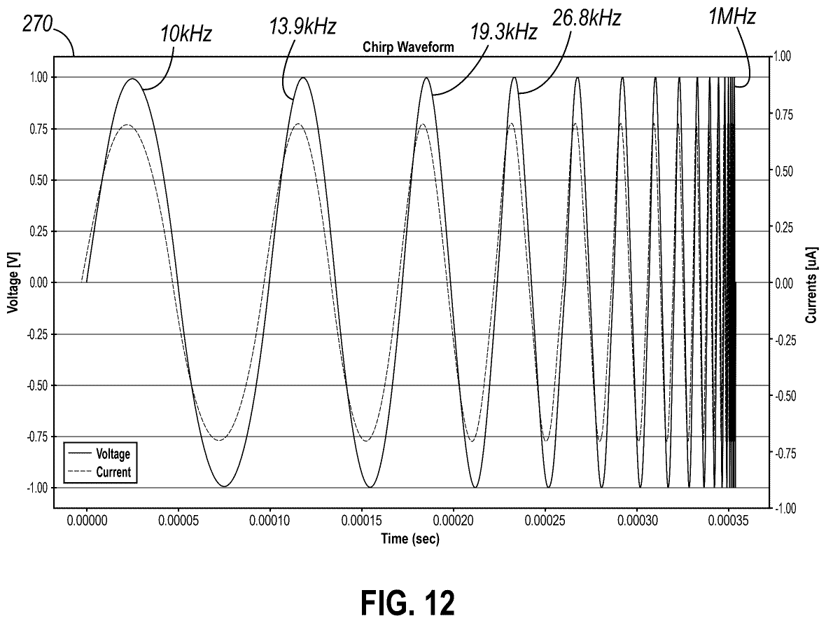

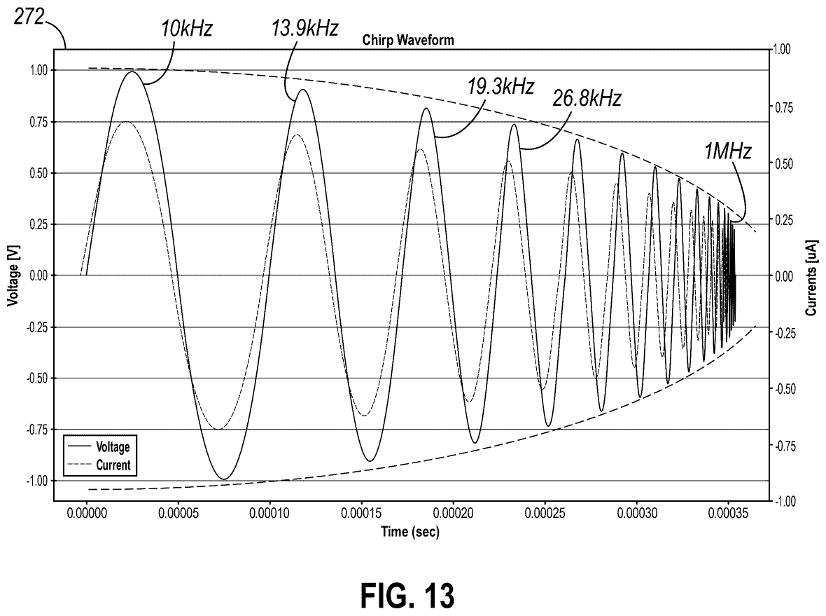

In a further version, a “chirp” function can be used, such as shown in FIG. 12. As would be understood by one skilled in the art, a chirp wave can be an “up-chirp” (i.e., the frequency increases) or a “down-chirp” (i.e., the frequency decreases). Thus, stated differently, a chirp function is essentially an advanced form of FM (264). The chirp function shown in a sixth graph (270) shows a chirp waveform with increasing frequency (e.g., 10 kHz, 13.2 kHz, 19.3 kHz, 26.8 kHz, and 1 Mhz). FIG. 13 shows a seventh graph (272) depicting a chirp function with the same frequencies as shown in FIG. 12, but with a decreasing amplitude. Additional features associated with electrical circuits and measurements of tissue are described in U.S. patent application Ser. No. 17/854,306, entitled “Electrosurgical Instrument for Applying Non-Therapeutic RF Signals,” filed Jun. 30, 2022, and published as U.S. Pat. Pub. No. 2024/0000499 on Jan. 4, 2024, the disclosure of which is incorporated by reference herein, in its entirety.

### III. ILLUSTRATIVE USE OF TISSUE IMPEDANCE SENSING DURING USE OF ELECTROSURGICAL INSTRUMENT

As mentioned above, end effector (180) is configured to apply non-therapeutic bipolar radiofrequency (RF) energy to securely clamped tissue in order to identify and/or verify that the correct tissue is presented in the end effector such that a therapeutic RF energy can be applied to seal or weld tissue. In the current example, electrosurgical instrument (100) is electrically coupled to waveform generator (200), which is capable of delivering both therapeutic and non-therapeutic energy to end effector (180). Non-therapeutic energy may also be referred to as subtherapeutic energy.

Further, one or more electrodes (194, 196) of end effector (180), which are configured to engage tissue when end effector (180) clamps tissue in accordance with the description herein, are operatively connected to sensor devices to determine the impedance phase angle and magnitude of the electrical circuit which non-therapeutic energy is transmitted through (e.g., such an electrical circuit may be partially formed by electrodes (194, 196) and the tissue captured therebetween). A processor (e.g., control unit (102)) may receive this impedance information, utilize the impedance information, and/or relays such impedance information to suitable electronic devices for use during or after a surgical procedure.

### A. Illustrative Application of Non-therapeutic RF Energy Sensing During Illustrative Use of Surgical Instrument to Seal and Cut Tissue

In some instances, it may be desirable to transmit non-therapeutic energy to one or more electrodes (194, 196) and measure the resulting impedance phase angle and magnitude of the corresponding electrical circuit during moments other than when tissue is securely clamped by end effector (180). Applying non-therapeutic energy to one or electrodes (194, 196) and measuring the resulting impedance phase angle and magnitude of the corresponding electrical circuit during other moments of a surgical procedure may provide other insights that may be utilized to inform a surgeon and/or logged into data storage for later use. Applying non-therapeutic energy to one or electrodes (194, 196) at multiple frequencies and measuring the resulting impedance phase angles and magnitudes for each frequency of the corresponding electrical circuit may be referred to as a frequency sweep, subtherapeutic energy frequency sweep, non-therapeutic energy frequency sweep, subtherapeutic impedance sensing, and/or non-therapeutic impedance sensing.

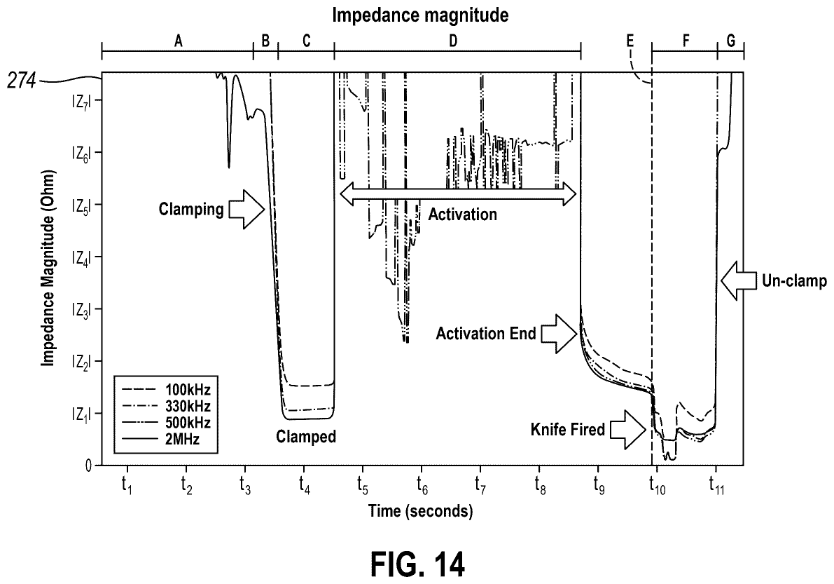

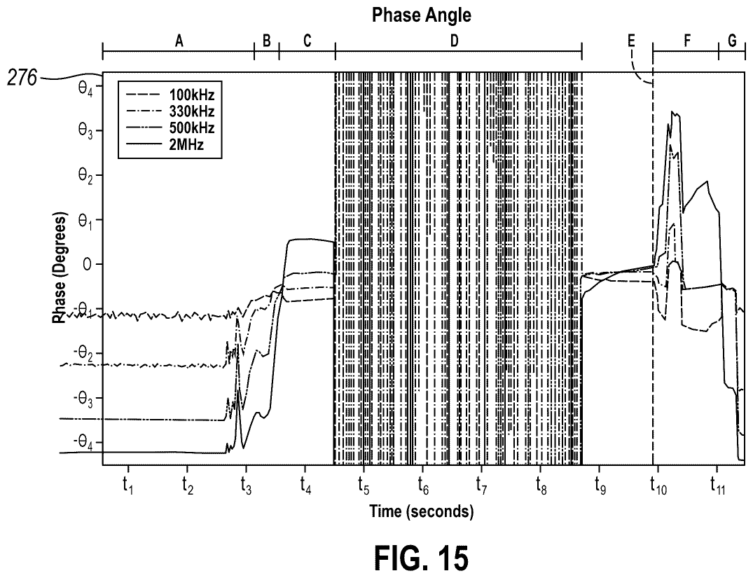

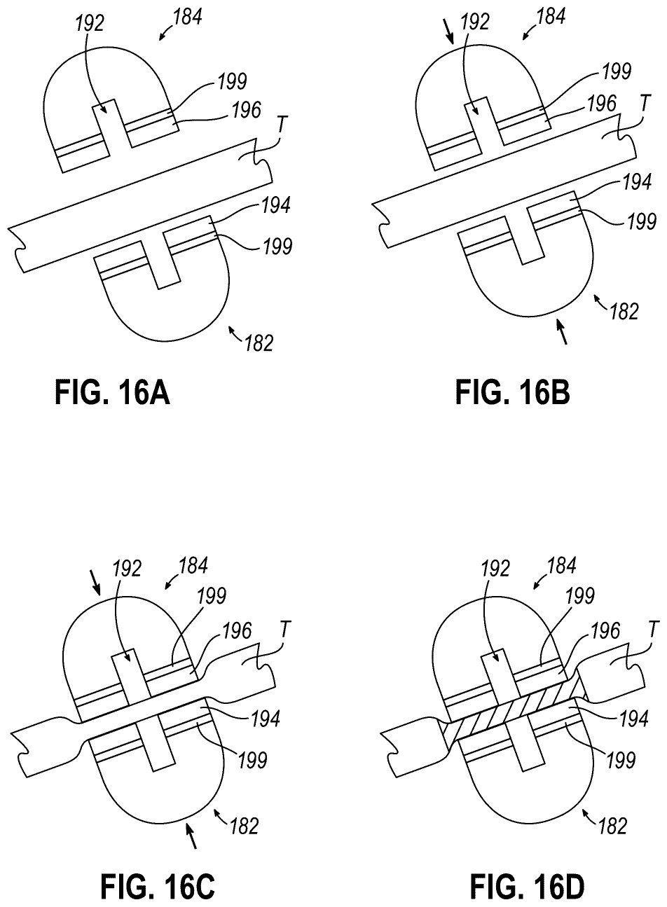

FIGS. 14 and 15 show an eighth graph (274) and ninth graph (276), respectively, depicting measurements of impedance magnitudes (FIG. 14) and phase angles (FIG. 15) measured within an electrical path at least partially formed by one or more electrodes (194, 196) in response to transmitted non-therapeutic energy at various frequencies (e.g., an illustrative non-therapeutic frequency sweep) during illustrative use of end effector (180). FIGS. 16A-16G show an illustrative use of end effector (180) to seal and sever tissue during the measured time frames and moments displayed in the graphs of FIGS. 14-15. As can be seen in FIGS. 14-15, non-therapeutic energy is transmitted and measured at various frequencies, which in the current example are 100 kHz, 330 kHz, 500 kHz, and 2 MHz. It should be understood that any suitable frequency values, range(s) of frequencies, number of frequencies, etc., may be utilized as would be apparent to one skilled in the art in view of the teachings herein.

As will be described in greater detail below, different frequencies in the frequency sweep may react differently (i.e., measured phase angle and/or magnitude, measured rate of change in phase angle and/or magnitude) in response to the different conditions of illustrative use of end effector (180). These variations of characteristics in the frequency sweep may be utilized by a processor receiving such information (e.g., control unit (102)) to identify notable moments of illustrative use of end effector (180), notify a surgeon of such identified moments, and/or segment data collected during illustrative use of end effector (180) using the identified notable moments for data logging purposes. A notable moment of illustrative use of end effector (180) may be moment of interest, either for the surgeon during actual use of instrument (100), or for others analyzing data accumulated during illustrative use of instrument (100). Such notable moments may be utilized to segment data collected during use of instrument (100). For example, non-limiting examples of a notable moment may be when jaws (182, 184) pivot from the open position toward the clamped position, when jaws (182, 184) reach the clamped position, when tissue grasped by end effector (180) is ready to receive therapeutic energy, when it is suitable for knife member (176) to be fired, when knife member (176) is fired, when knife member (176) is retracted after firing, when jaws (182, 184) are opened to release tissue, etc.

It should be understood that a frequency sweep of non-therapeutic energy being applied to one or more electrodes (194, 196) may be applied from the start of section (A) (i.e., jaws (182, 184) in the open position prior to engaging and clamping tissue) all the way to the end of section (G) (i.e., jaws (182, 184) unclamping tissue after being sealed and severed). However, it should also be understood that frequency sweeps of non-therapeutic energy may be temporarily paused or halted during any suitable time of illustrative use of end effector (180) between and including time frame (A) and time frame (G). For illustrative purposes, frequency sweeps may be temporarily paused during activation of end effector (180) with therapeutic energy during time frame (D).

Time frame (A) shown in FIGS. 14-15 corresponds with the moment shown in FIG. 16A during a non-therapeutic frequency sweep. In particular, during time frame (A), jaws (182, 184) are in the open position such that tissue (T) is not in contact with electrodes (194, 196). With tissue (T) not engaged with (i.e., in contact with) electrodes (194, 196), the impedance magnitude measured (as shown in FIG. 14) is substantially high, even above the highest impedance value |Z7| shown in FIG. 14. This measured impedance magnitude may be due in part to the fact electrodes (194, 196) are essentially forming an open circuit. Additionally, as shown in FIG. 15, the measured phase angles for each frequency in the frequency sweep are substantially consistent. The processor receiving such information may utilize the received measurements from the frequency sweep, as well as the known trends of impedance magnitudes and phase angles during illustrative use of instrument (100), to identify that jaws (182, 184) are substantially open, and yet to be closed.

Time frame (B) shown in FIGS. 14-15 corresponds with the movement of jaws (182, 184) being closed in preparation of clamping tissue (T) during a non-therapeutic frequency sweep, as illustrated in FIG. 16B. During time frame (B), jaws (182, 184) are transitioning from the open position toward the closed position in order to clamp tissue. Therefore, during time frame (B), electrodes (194, 196) may transition from not contacting tissue (T), to initially contacting tissue, to initially clamping tissue (T) as shown in FIG. 16C. With tissue (T) initially being engaged with electrodes (194, 196), the impedance magnitude measured (as shown in FIG. 14) sharply decreases (e.g., at a high negative slope/rate of change) from its high value in time frame (A) toward |Z1|. Additionally, at the initial start of closing jaw (182, 184) the higher frequencies of the frequency sweep have measured phase angles with a sharp increase and subsequent decrease, resembling a spike in change, during time frame (B). Subsequently, as jaws (182, 184) move toward each other, the phase angle of each frequency in the frequency sweep tends to generally rise from a negative phase angel toward zero and/or a positive phase angle, with the larger frequency (in this instance, 2 MHz) raising faster (and eventually surpassing) the lower frequency phase angles. The processor receiving such information may utilize the received measurements from the frequency sweep, as well as the known trends of impedance magnitudes and phases angle during illustrative use of instrument (100), to identify that jaws (182, 184) have been initially closed toward the clamped configuration with tissue located between electrodes (194, 196).

Time frame (C) shown in FIGS. 14-15 corresponds with jaws (182, 184) remaining clamped onto tissue (T) during a non-therapeutic frequency sweep prior to activating electrodes (194, 196) with therapeutic energy, as illustrated in FIG. 16C. Jaws (182, 184) may clamp onto tissue (T) for a suitable amount of time prior to activating electrodes (194, 196). Such clamping of tissue (T) may force fluids out of tissue (T) (e.g., a milking effect), altering the electrotonic properties of such tissue (T); which may in turn make tissue (T) more amenable to being suitably sealed with therapeutic RF energy. With tissue (T) clamped between electrodes (194, 196), the impedance magnitude measured prior to therapeutic activation of electrodes (194, 196) (as shown in FIG. 14) remains relatively constant across the frequency sweep around |Z1| and |Z2|. After the phase angles generally increase as shown at the end of time frame (B), the phase angle measurements begin to level out (i.e., remain relatively constant), which may indicate that tissue (T) is suitably clamped and prepared to receive therapeutic energy. The processor receiving such information may utilize the received measurements from the frequency sweep, as well as the known trends of impedance magnitudes and phase angles during illustrative use of instrument (100), to identify that jaws (182, 184) are clamping tissue (T) and the clamped tissue (T) is ready to receive therapeutic energy in accordance with the description herein.

Time frame (D) shown in FIGS. 14-15 corresponds with activation of electrodes (194, 196) to thereby apply therapeutic energy to tissue (T) to thereby seal tissue (T), as illustrated in FIG. 16D, during a non-therapeutic frequency sweep. In some instances, the electrodes configured to perform a non-therapeutic frequency sweep are different from the electrodes (194, 196) configured to apply therapeutic energy, such that both may occur simultaneously.

With therapeutic energy being delivered to tissue (T) via electrodes (194, 196), the impedance magnitude of the various frequencies of the frequency sweep may quickly rise and decline (or oscillate) between various suitable values. Simultaneously, the impedance phase angles of the various frequencies of the frequency sweep also quickly rise and decline between various suitable negative and positive phase angles. The processor receiving such information may utilize the received measurements from the frequency sweep, as well as the known trends of impedance magnitudes and phase angles during illustrative use of instrument (100), to identify that therapeutic energy is being delivered to tissue (T) via electrodes (194, 196).

In some instances, when electrodes (194, 196) transmit therapeutic energy to tissue (T), transmission of non-therapeutic energy may be halted or temporarily paused. Therefore, the processor receiving information from non-therapeutic RF energy frequency sweeps may not obtain corresponding impedance information while end effector (180) performs its therapeutic energy delivery algorithm. In some instances, the processor receiving information from the frequency sweep of non-therapeutic energy may be aware that electrodes (194, 196) are activated with therapeutic energy. Therefore, in instances where non-therapeutic RF energy is temporarily paused during the delivery of therapeutic RF energy, after the therapeutic energy delivery algorithm is completed, the non-therapeutic RF energy may continue to be transmitted to one more electrodes (194, 196) and measured such that processor continues to receive such information in accordance with the description herein. Of course, frequency sweeps may be paused and resumed using any suitable algorithm(s) as would be apparent to one skilled in the art in view of the teachings herein.

After end effector (180) completes its therapeutic RF energy activation cycle, as indicated by the end of time frame (D), tissue (T) captured between jaws (182, 184) may be sealed, as shown in FIG. 16D. Once sealing is completed and therapeutic RF energy activation ceases to be delivered to tissue (T), it may be desirable to wait a suitable amount of time before severing tissue (T) with knife member (176) in accordance with the description herein. Waiting a suitable amount of time between the end of a therapeutic activation cycle and the firing of knife member (176) may provide a better quality of severed and sealed tissue. For example, waiting a suitable amount of time may allow tissue to cool down to a desirable temperature that allows knife member (176) to more easily/reliably sever tissue.

As shown between time frame (D) and moment (E) in FIGS. 14-15 during a non-therapeutic frequency sweep, the impedance magnitude of the frequency sweep may generally decline with a concave slope to values at or around |Z1| and |Z2|. Additionally, as shown in FIG. 15, the phase angle of the 2 MHz frequency may gradually increase from a negative phase angle toward a zero-degree phase angle such that the 2 MHz frequency approaches the zero-degree phase angle. The processor receiving such information may utilize the received measurements from the frequency sweep, as well as the known trends of impedance magnitudes and phase angles during illustrative use of instrument (100), to identify that a suitable amount of time has passed for knife member (176) to be fired to sever tissue. For example, the processor may generate an indication signal that assists in indicating to a user that knife member (176) is ready to be fired when a phase angle of a suitable frequency of the frequency sweep approaches and/or reaches a zero-degree phase angle.

Next, as shown at moment (E) in FIGS. 14-15, a user may fire knife member (176) in accordance with the description herein during a non-therapeutic frequency sweep, as illustrated in FIG. 16E, therefore severing tissue (T) that was recently sealed via therapeutic RF energy. With knife member (176) fired, as shown in FIG. 14, the impedance magnitude of each frequency declines further with a sharp negative slope. Additionally, as shown in FIG. 15, the phase angles of each frequency may spike upwards into a positive phase angle and then back downwards while knife member (176) is fired or immediately after knife member (176) is fired. The processor receiving such information may utilize the received measurements from the frequency sweep, as well as the known trends of impedance magnitudes and phase angles during illustrative use of instrument (100), to identify that knife member (176) has been fired.

Next, as shown in time frame (F) in FIGS. 14-15, knife member (176) is retracted during a non-therapeutic frequency sweep while jaws (182, 184) remain clamped onto tissue (T) that has been sealed and severed in accordance with the description herein, as illustrated in FIG. 16F. With recently sealed tissue (T) severed and knife member (176) retracted, the impedance magnitude of each frequency may fluctuate near a relatively lower impedance magnitude value, such as |Z1| shown in FIG. 14. Additionally, as shown in FIG. 15, the phase angles of each frequency may remain at a relatively stable value or range of values. The processor receiving such information may utilize the received measurements from the frequency sweep, as well as the known trends of impedance magnitudes and phase angles during illustrative use of instrument (100), to identify that knife member (176) has been retracted while jaws (182, 184) remain closed.

Next, as shown in time frame (G) in FIGS. 14-15, jaws (182, 184) may be opened to release the recently sealed and severed tissue (T) during a non-therapeutic frequency sweep, as illustrated in FIG. 16F. With jaws (182, 184) opened to release tissue (T), tissue (T) is no longer engaged with electrodes (194, 196). With tissue (T) not engaged with (i.e., in contact with) electrodes (194, 196), the impedance magnitudes measured (as shown in FIG. 14) raises at relatively fast rates and returns to substantially high values, even above the highest impedance value |Z7| shown in FIG. 14. This measured impedance magnitude may be due in part to the fact electrodes (194, 196) are essentially forming an open circuit. Additionally, as shown in FIG. 15, the measured phase angles for each frequency in the frequency sweep returns to the substantially consistent levels prior to initially grasping tissue (T), similar to those in time frame (A). The processor receiving such information may utilize the received measurements from the frequency sweep, as well as the known trends of impedance magnitudes and phase angles during illustrative use of instrument (100), to identify that jaws (182, 184) are substantially open, and have released tissue (T).

### B. Illustrative Detection, Notification, and Logging of Notable Events

### Using Non-Therapeutic RF Energy Sensing

As mentioned above, known trends in behavior of impedance magnitudes and phase angles of a frequency sweep during illustrative use of end effector (180) may be utilized by a processor (e.g., control unit (102)) receiving current (e.g., live) frequency sweep information (e.g., comparing/analyzing known trends v. live frequency sweep data) to identify notable moments, notify a surgeon of such identified moments, and/or segment data collected during illustrative use of end effector (180) for data logging purposes.

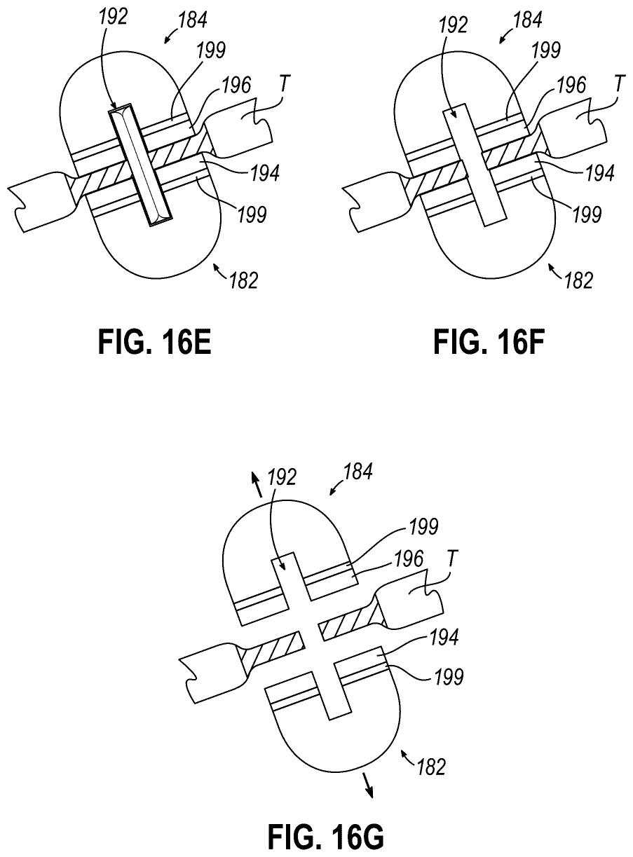

FIG. 17 shows an illustrative method (300) in which a control unit (102) and/or waveform generator (200) (or any other suitable processor, memory unit, suitable circuitry, etc.) may utilize non-therapeutic energy frequency sweeps in order to detect, display, and/or log various notable events during illustrative use of surgical system (98) in accordance with the description herein. First, during one illustrative use, instrument (100) may be suitably coupled to waveform generator (200). In the current example, control unit (102) of instrument (100) may be in suitable communication with waveform generator (200) (and other suitable electronic instruments, such as visual screens, data collecting devices associated with waveform generator (200), etc.).

Next, during illustrative use of instrument (100), method (300) may include the suitable processing unit (e.g., control unit (102) or suitable components of waveform generator (200)) determining (302) if electrodes (194, 196) are activated with therapeutic energy. If the answer is yes, the suitable processing unit may wait (305) for the activation cycle of therapeutic energy to end. If the suitable processing unit determines that electrodes (194, 196) are not activated with therapeutic energy, the processing unit may instruct instrument (100) and/or waveform generator (200) to apply (304) subtherapeutic impedance sensing. In other words, the processing unit may instruct instrument (100) and/or waveform generator (200) to initiate a non-therapeutic frequency sweep.

In the current example, method (300) includes waiting to initiate a non-therapeutic frequency sweep until electrodes (194, 196) are not activated with therapeutic energy. However, this is merely illustrative. In some examples, a non-therapeutic frequency sweep may be conducted even if therapeutic energy is being communicated to electrodes (194, 196). In some instances, the electrodes configured to perform a non-therapeutic frequency sweep are different from the electrodes (194, 196) configured to apply therapeutic energy, such that both may occur simultaneously.

Next, the suitable processing unit utilizes the information received from the non-therapeutic frequency sweep (i.e. applying subtherapeutic impedance sensing to electrodes (194, 196) at various frequencies), as well as the known trends of impedance magnitudes and phase angles during illustrative use of instrument (100), to determine if clamping is detected (306), to determine if knife firing is detected (310), to determine if jaw opening is detected (314) (e.g., openings jaws (182, 184) after suitable firing of knife member (176) and/or opening jaws (182, 184) without applying therapeutic energy and/or firing knife member (176) in order to regrasp tissue), or to determine any other suitable notable event as would be apparent to one skilled in the art. Suitable processing unit may utilize information from the non-therapeutic frequency sweep in substantially similar manner described above in order to identify such notable events.

Next, if the suitable processing unit detects a noteworthy event (306, 310, 314) at least partially in response to information from the frequency sweep, the processing unit may then display and/or log (308, 312, 316) such a notable event and the time at which the notable event occurred. If displayed, such a display may be visual. In some instances, in addition to a visual display (or as an alternative); an audible, tactile, and/or suitable combination thereof may be utilized. This information may be used to indicate to a surgeon during a surgical procedure that such an event has been detected.

The detected notable events may be logged in order to segment data stored during the use of the surgical event (e.g., segmenting the moment when jaws (182, 184) are open and when jaws (182, 184) are closed). Additionally, the notable events may be logged for any other suitable purpose as would be apparent to one skilled in the art in view of the teachings herein. For example, these notable events may be used to determine how efficient a surgical procedure was performed (e.g., comparing how many times tissue was clamped v. how many times knife was fired).

If a notable event is not detected (306, 310, 314), then the processing unit may return to the beginning of the method (300) and determine if electrodes (194, 196) are activated. Additionally, or alternatively, the processing unit may continue to monitor the non-therapeutic frequency sweeps until a notable event is detected.

### C. Illustrative Instructions Generated Using Non-Therapeutic RF Energy

Information obtained from non-therapeutic frequency sweeps during the use of instrument (100) may be used for suitable purposes other than detecting notable events, communicating such detection of notable events, and/or logging data utilizing detected notable events. For example, as mentioned above, and as will be described in greater detail below, surgical system (98) may utilize information from non-therapeutic frequency sweeps to instruct surgeon to perform a task at a suitable time during the procedure (e.g., firing knife, activating electrodes with therapeutic energy, opening jaws after severing tissue, etc.).

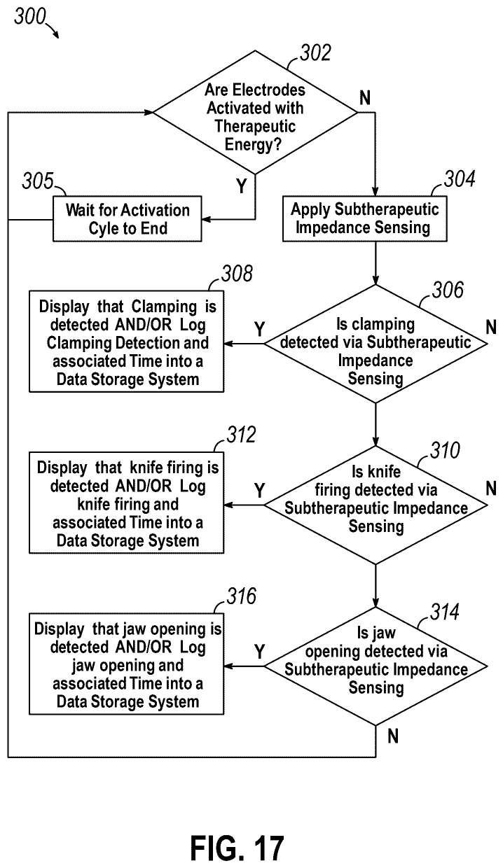

FIG. 18 shows an illustrative method (350) in which control unit (102) and/or waveform generator (200) (or any other suitable processor, memory unit, suitable circuitry, etc.) may utilize non-therapeutic energy frequency sweeps in order to generate signals indicting a surgeon to perform a task (in the current example, firing knife member (176)). As mentioned above, knife member (176) may be fired after a suitable amount of time from when end effector (180) completes a therapeutic energy activation cycle in order to seal tissue (T). Therefore, method (350) may include starting a therapeutic energy activation cycle (352) and ending the therapeutic energy activation cycle (354) in order to suitably seal tissue (T) in accordance with the teachings herein.

In the current example, after the therapeutic energy activation cycle has ended (354), control unit (102) and/or waveform generator (200) may activate end effector (180) to perform/apply (356) subtherapeutic impedance sensing (i.e., a frequency sweep). In some instances, the electrodes configured to perform a non-therapeutic frequency sweep are different from the electrodes (194, 196) configured to apply therapeutic energy, such that both may occur simultaneously. In such instances, performing/applying (356) subtherapeutic impedance sensing may be initiated prior to ending the therapeutic energy activation cycle (354).

As mentioned above, after a therapeutic energy activation cycle (354) is performed to seal tissue, the phase angle of the 2 MHz frequency (or another suitable frequency) may gradually increase from a negative phase angle toward a zero-degree phase angle such that the 2 MHz frequency approaches the zero-degree phase angle. As also mentioned above, the processor (e.g., control unit (102) and/or waveform generator (200)) receiving such information may utilize the received measurements from the frequency sweep (e.g., compare/analyze the received information from the known trends of impedance magnitudes and phase angles during illustrative use of instrument (100)), and identify that a suitable amount of time has passed for knife member (176) to be fired to sever tissue.

Therefore, during the subtherapeutic sensing (356) of method (350), control unit (102) and/or waveform generator (200) may monitor the phase angle change (358) of select frequencies and utilize the known trends of impedance magnitudes and phase angles during illustrative use of instrument (100) to detect (360) when slope of the phase angle of suitable frequencies reaches a threshold (e.g., approaching the zero-degree phase angle with a suitable slope). Such a detection (360) may indicate that a suitable amount of time may have passed since the end of the therapeutic energy activation cycle for knife member (176) to be fired to sever tissue. Therefore, in response to such detection (360), control unit (102) and/or waveform generator (200) may generate (3652) a signal indicating to a surgeon to proceed to firing knife member (176). Such a signal may be auditory, visual, tactile, or any other suitable signal as would be apparent to one skilled in the art in view of the teachings herein.

While the current illustrative method (350) includes utilizing subtherapeutic sensing, as well as the known trends of impedance magnitude and phase angle during illustrative use of instrument (100), to determine when to instruct a surgeon to fire knife member (176); surgical system (98) may utilize subtherapeutic sensing to determine when to instruct a surgeon to perform other tasks associated with the use of instrument (100). Non-limiting illustrative examples include when to activate electrodes with therapeutic energy, when to opening jaws after severing tissue, and/or any other suitable instruction as would be apparent to one skilled in the art in view of the teachings herein.

As mentioned above, the various measured impedance magnitudes and phase angles in a frequency sweep tend to change in response to jaws (182, 184) transitioning from the open position toward the closed position in order to clamp tissue. As mentioned above, the processor receiving such information may utilize the received measurements from the frequency sweep, as well as the known trends of impedance magnitude and phase angle during illustrative use of instrument (100), to identify when jaws (182, 184) have been initially closed toward the clamped configuration with tissue located between electrodes (194, 196). In some instances, it may be desirable to utilize the information from impedance sensing, as well as the known trends of impedance magnitude and phase angle during illustrative use of instrument (100), in order to approximate, track, and log the jaw gap (i.e., a jaw aperture).

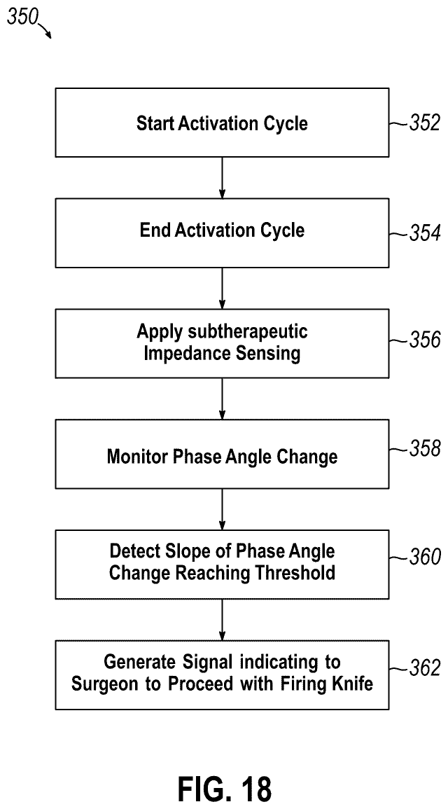

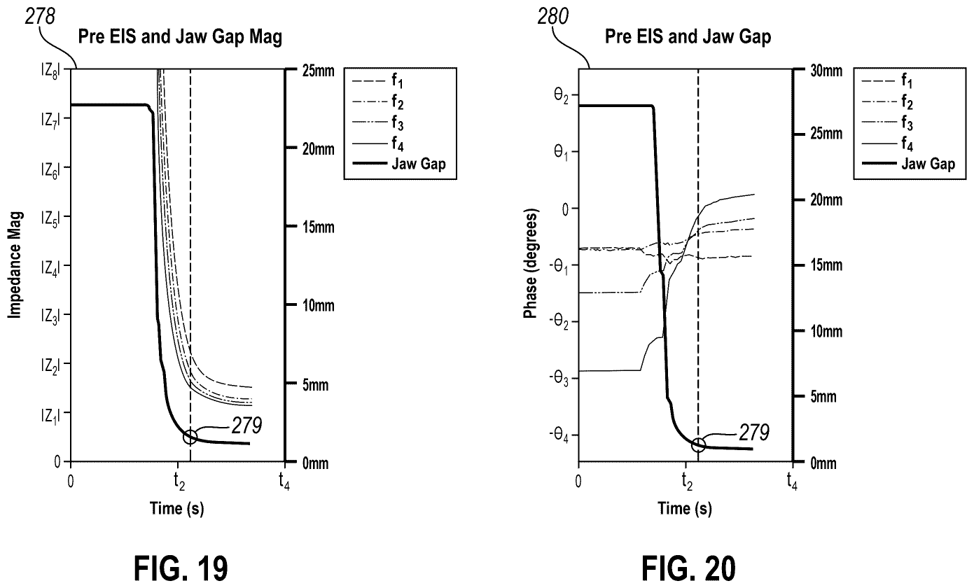

FIGS. 19 and 20 show a tenth graph (278) and an eleventh graph (280) that provides visual representations of the gap between jaws (182, 184) (i.e., a jaw gap), measured in millimeters, as jaws are being closed over a time period, against the measured impedance magnitude (FIG. 19) and phase angel (FIG. 20) provided from non-therapeutic impedance sensing while jaws are being closed over the same time period.

### D. Illustrative Jaw Aperture Approximation Using Non-Therapeutic RF Energy Sensing

As mentioned above, while jaws (82, 184) are transitioning from the open position toward the closed position, electrodes (194, 196) may transition from not contacting tissue (T), to initially contacting tissue (T), to initially clamping tissue (T). Initially clamping tissue (T) is illustrated at location (279). In response, as further illustrated in FIG. 19, the impedance magnitude measured (as shown in FIG. 14) sharply decreases (e.g., at a high negative slope/rate of change) from a high value toward |Z1|. Once clamping occurs at location (279), the rate of change of impedance magnitude levels out to a more consistent value. Simultaneously, as further illustrated in FIG. 20, as jaws (182, 184) move toward each other, the phase angle of each frequency in the frequency sweep tends to generally rise from a negative phase angel toward zero and/or a positive phase angle, with the larger frequency (in this instance, (f4)) rising faster (and eventually surpassing) the lower frequency phase angles. Similarly, once clamping occurs at location (279), the rate of change of phase angle measurements levels out to more consistent values.

The processor receiving such information may utilize the received measurements from the subtherapeutic impedance sensing (i.e., frequency sweep) while jaws (182, 184) move relative to each other, as well as the known trends of impedance magnitude and phase angle during illustrative use of instrument (100), in order to approximate the jaw gap during illustrative use of instrument (100). The information related to the approximated jaw gap during illustrative use may be utilized to provide various information. For example, between activating jaws (182, 184) with therapeutic energy, approximation of jaw gap using subtherapeutic impedance sensing may provide data related to the number of times tissue was grasped versus the number of times end effector (180) was fired, the number of times jaws (182, 184) were repositioned prior to firing, etc. As another example, using subtherapeutic impedance sensing to approximate jaw gap may be used to provide metrics on tissue compression (changes in electrical signals after clamping and pre-firing). As another example, using subtherapeutic impedance sensing to approximate jaw gap may be used to segment impedance signals utilized to inform energy delivery algorithms to only the periods just before or after activation. As another example, using subtherapeutic impedance sensing to approximate jaw gap may be used as a means of tracking device use for the purpose of segmenting and annotating video data.

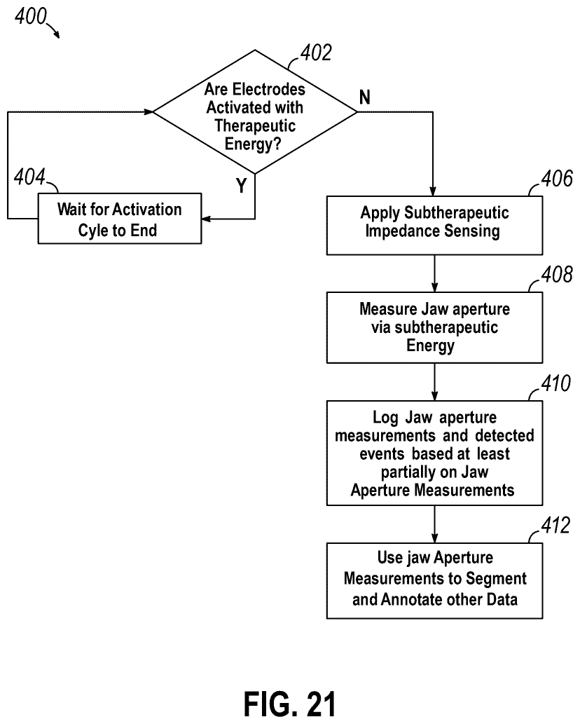

FIG. 21 shows an illustrative method (400) in which a control unit (102) and/or waveform generator (200) (or any other suitable processor, memory unit, suitable circuitry, etc.) may utilize non-therapeutic energy frequency sweeps, as well as the known trends of impedance magnitudes and phase angles during illustrative use of instrument (100), in order to approximate the jaw gap during illustrative use and to further leverage the jaw gap approximation to log the jaw gap measurement, detect events based at least partially on jaw gap measurements, and further use jaw gap measurement to segment or annotate other data collected by system (98) during illustrative use.

During illustrative use of instrument (100) in conjunction with method (400), a suitable processing unit (e.g., control unit (102) or suitable components of waveform generator (200)) determines (402) if electrodes (194, 196) are activated with therapeutic energy. If the answer is yes, the suitable processing unit may wait (404) for the activation cycle of therapeutic energy to end. If the suitable processing unit determines that electrodes (194, 196) are not activated with therapeutic energy, the processing unit may instruct instrument (100) and/or waveform generator (200) to apply (406) subtherapeutic impedance sensing. In other words, the processing unit may instruct instrument (100) and/or waveform generator (200) to initiate a non-therapeutic frequency sweep.

In the current example, method (400) includes waiting to initiate a non-therapeutic frequency sweep until electrodes (194, 196) are not activated with therapeutic energy. However, this is merely illustrative. In some examples, a non-therapeutic frequency sweep may be conducted even if therapeutic energy is being communicated to electrodes (194, 196). In some instances, the electrodes configured to perform a non-therapeutic frequency sweep are different from the electrodes (194, 196) configured to apply therapeutic energy, such that both may occur simultaneously.

Next, the suitable processing unit utilizes the information received from the non-therapeutic frequency sweep (i.e. applying subtherapeutic impedance sensing to electrodes (194, 196) at various frequencies), as well as the known trends of impedance magnitude and phase angle during illustrative use of instrument (100), to measure (408) the jaw aperture (i.e., jaw gap) via subtherapeutic energy (i.e., frequency sweep). In other words, the processing unit may utilize the measured impedance magnitude and phase angles provided in real-time, and compare those measurements to known trends in order to approximate/measure the jaw aperture.

The processing unit may further utilize the jaw aperture measurements as would be apparent to one skilled in the art in view of the teachings herein. In the current example, the processing unit uses the measured jaw aperture from subtherapeutic sensing in order to log and detect (410) events, such as clamping tissue, repositioning jaws (182, 184) to re-clamp tissue, etc. The processing unit may log and detect (410) events based at least partially on the jaw aperture measurements. Further, the processing unit may use (412) jaw aperture measurement from subtherapeutic sensing in order to segment an annotate (412) other data obtained while using system (98).

E. Illustrative End Effector with Jaw Gap Sensor and Non-Therapeutic RF Energy Sensing

As mentioned above, non-therapeutic energy may be applied to clamped tissue in order to help determine tissue characteristics, such as tissue type, tissue phase, tissue margin, and the like. In some instances, it me be desirable to supplement data obtained by subtherapeutic impedance sensing with additional data in order to provide more accurate information with regards to tissue (T) captured within end effector (180). For example, a thin piece of fat and a thick piece of muscle may provide similar impedance magnitudes and be confused due to the difference in tissue geometry. Knowing the jaw gap measurement could mitigate these risks by normalizing the impedance measurements based on the know jaw gap between jaws (182, 184).

As another example, in instances where jaws (182, 184) are moving toward each other, impedance measurements from frequency sweeps may change due to movement of jaw (182, 184) relative to each other, rather than from changes in the tissue (T) located between jaws (182, 184). However, in instances where jaws (182, 184) are not moving, but the impedance measurement from frequency sweeps are changing, then the changes in the impedance measurement may be stemming from the tissue itself in response to the application of pressure from jaws (182, 184) and/or the delivery of therapeutic RF energy to tissue from electrode (194, 196).

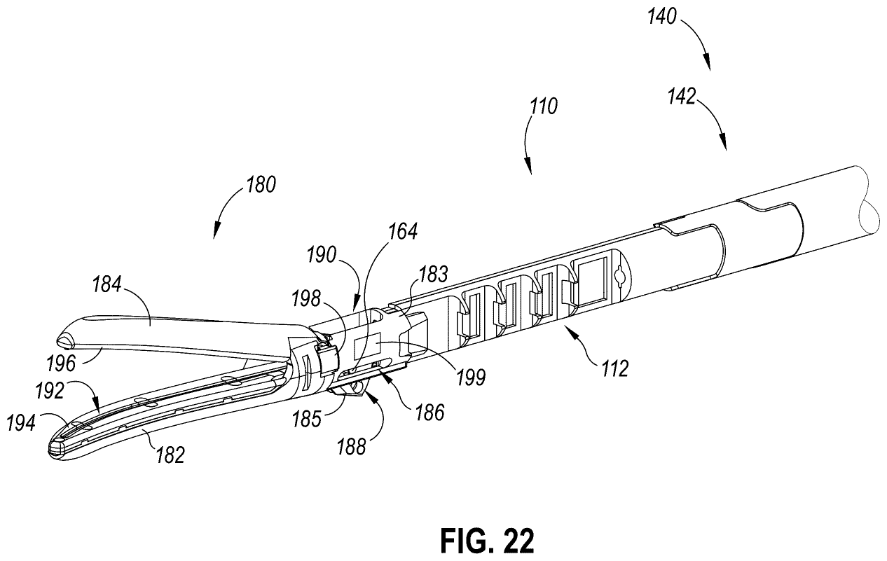

Therefore, having confirmation of the jaw gap between jaws (182, 184) in addition to impedance measurements from frequency sweeps may provide system (98) a more accurate representation of what end effector (180) is experiencing during illustrative use in accordance with the description herein. FIG. 22 show an illustrative alternative end effector (180′) that may be readily incorporated into instrument (100) described above, with differences elaborated below. Therefore, end effector (180′) is substantially similar to end effector (180) described above. However, end effector (180′) includes a suitable jaw gap sensor (199) that is configured to accurately measure the jaw gap between electrodes (194, 196) and communicate the measured jaw gap to control unit (102) such that control unit (102) may store the measured jaw gap over illustrative use of instrument (100) and utilize the measured jaw gap in accordance with the description herein. Jaw gap sensor (199) may include any suitable components as would be apparent to one skilled in the art in view of the teachings herein. For example, jaw gap sensor (199) may include an inclinometer, an LVDT, a Hall Effect sensor, etc.

Control unit (102) may utilize the measured jaw gap in conjunction with the impedance measurement from the non-therapeutic frequency sweeps in order to provide a more accurate analysis of tissue (T) being grasped and manipulated by jaws. Therefore, control unit (102) may utilize information from non-therapeutic frequency sweeps in accordance with the description herein, but supplement the use with the additional information of known tissue gap from jaw gap sensor (199).

As one example, control unit (102) may normalize impedance measurements from frequency sweeps based on the jaw gap measurement provided by sensor (199). Control unit (102) may determine if changes in the impedance measurements are due to movement of jaws (182, 184) relative to each other, changes in tissue characteristics from grasped tissue (T), or some combination of both (which may occur during the milking effect). In response to determinations of tissue characteristics, control unit (102) may identify a tissue property, identify a tissue action, or alter an algorithm to therapeutically seal tissue with RF energy.

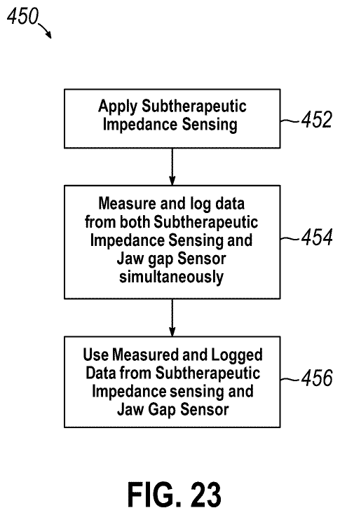

FIG. 23 shows an illustrative method of using system (98) having end effector (180′) with subtherapeutic impedance sensing in conjunction with jaw gap sensor (199). First, system (98) may apply (452) subtherapeutic impedance sensing to end effector (180′) in accordance with the description herein. It should be understood that jaw gap sensor (199) may be measuring the jaw gap between jaws (182, 184) and communicating such measurement to suitable components of system (98).

Next, system (98) may measure and log (454) data from both the subtherapeutic impedance sensing and the jaw gap sensor (199) simultaneously (454). With data available, system (98) may then use (456) measured and logged data from the subtherapeutic impedance sensing and the jaw gap sensor (199) in accordance with the description herein. For example, system (98) may normalize (or otherwise filter) data obtained from subtherapeutic impedance sensing based on the jaw gap measured by jaw gap sensor (199). As another example, system (98) may filter data obtained from subtherapeutic impedance sensing based on a measured rate of change (e.g., speed) of jaws (182, 184) moving relative to each other.

F. Illustrative End Effector with Jaw Gap Sensor, Jaw Clamp Sensor, and Non-Therapeutic RF Energy Sensing

In some instances, in addition to having a jaw gap sensor (199) used in conjunction with subtherapeutic impedance sensing in order to provide more accurate information with regards to tissue (T) captured between end effector (180), it may be desirable to identify the moment when jaws (182, 184) have reached the clamped position to thereby clamp tissue (T). Knowing when tissue (T) has been initially fully clamped by jaws (182, 184), in conjunction with both subtherapeutic impedance sensing and a jaw gap sensor, may also provide more accurate information with regards to tissue (T) captured between end effector (180).

For example, knowing when jaws (182, 184) have fully clamped tissue may help identify the zone between clamping and sealing in order to analyze impedance signals for tissue identification. Further, knowing the jaw gap may allow system (98) to normalize impedance measurements based on the distance between electrodes (194, 196). Identifying the point when jaws (182, 184) are fully clamped on tissue can be used to segment/isolate analysis of impedance signals and jaw gap measurement from when the clamping is initially achieved until electrodes (194, 196) activation, which may provide a more accurate analysis of data obtained from impedance sensing and jaw gap measurements.

Additionally, the change in jaw gap between the movement of jaws (182, 184) reaching the fully clamped position and subsequently being activated with therapeutic energy may be utilized in conjunction with subtherapeutic tissue sensing in order to better understand the strain-response of tissue following the clamp load. The changes in the electrical signal from subtherapeutic sensing may reflect compression changing the tissue thickness (and therefore jaw aperture), as well as a change in physical properties of the tissue (such as the milking effect).

Having data related to when clamping is achieved, what the jaw aperture is, as well as subtherapeutic sensing data, may allow for tissue differentiation. For example, when a properly skeletonized vessel is suitably grasped between jaws (182, 184), and jaws are clamped, the change in impedance magnitude over time may be generally smooth (as shown in FIG. 19). However, if a bundle of vessels is undesirably grasped between jaws (182, 184), and jaws (182, 184) are subsequently clamped, the impedance magnitude may be relatively higher and have deviations in value that are less smooth (i.e., having spikes in value). Knowing when jaws (182, 184) reach the fully clamped positions may allow for such subtherapeutic impedance trends to be utilized to determine if the tissue (T) grasped in a properly skeletonized vessel or an undesirable bundle of vessels.

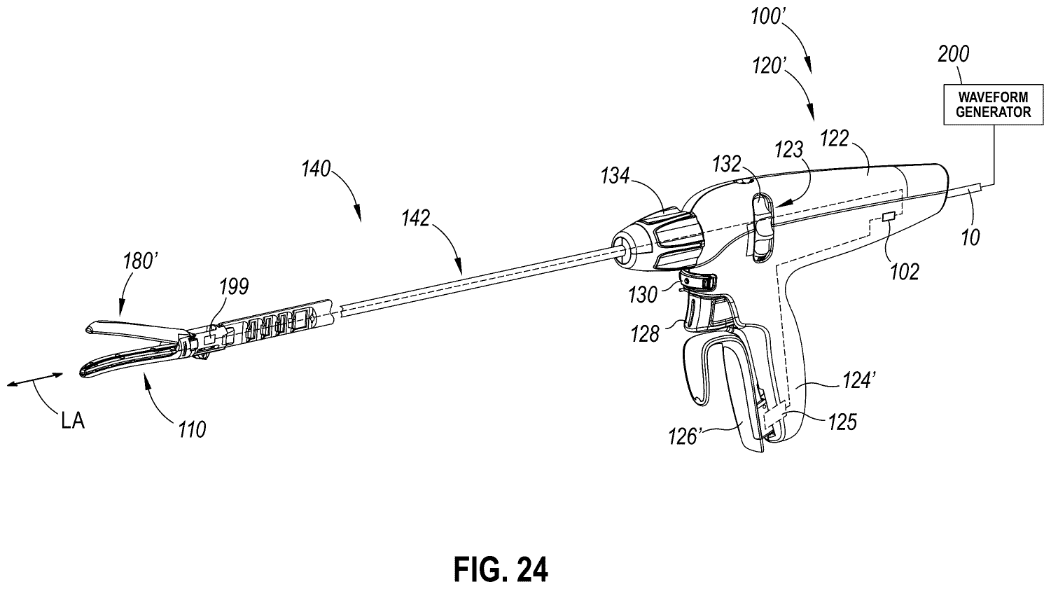

FIG. 24 shows an illustrative electrosurgical instrument (100′) that may be readily incorporated into surgical system (98) described above, in replacement of instrument (100). Therefore, instrument (100′) is substantially similar to instrument (100) described above, with differences elaborated herein.

Instrument (100′) includes end effector (180′) instead of end effector (180). Therefore, instrument (100′) includes jaw gap sensor (199) that configured to measure the jaw gap between jaws (182, 184) during illustrative use in accordance with the description herein and communicate the measured jaw gap to control unit (102). Additionally, instrument (100′) includes a jaw clamp sensor (125) in communication with control unit (102). Jaw clamp sensor (125) is configured to determine when jaws (182, 184) are in the fully clamped position. Further, jaw clamp sensor (125) is in communication with control unit (102) such that jaw clamp sensor (125) may communicate to control unit (102) when jaws (182, 184) are fully clamped.

In the current example, jaw clamp sensor (125) is associated with jaw closure trigger (126) and pistol grip (124). As mentioned above, jaw closure trigger (126) is configured to pivot relative to pistol grip (124) to actuate jaws (182, 184) between an open position and the fully clamped position. Jaw clamp sensor (125) is suitably attached to trigger (126) and grip (124) such that sensor (125) is configured to determine when jaws (182, 184) are fully clamped based on the position of jaw closure trigger (126) and pistol grip (124) relative to each other. Jaw clamp sensor (125) may include any suitable structures as would be apparent to one skilled in the art in view of the teachings herein. For example, jaw clamp sensor (125) may include a switch that is activated when trigger (126) and grip (124) reach a position associated with jaws (182, 184) being fully clamped.

As mentioned above, control unit (102) is in communication with both jaw gap sensor (199) and jaw clamp sensor (125). Therefore, control unit (102) may receive data from subtherapeutic RF energy sensing (i.e., frequency sweeps), from jaw gap sensor (199), and from jaw clamp sensor (125). As will be described in greater detail below, control unit (102) (or any other suitable processing device) may utilize data obtained from subtherapeutic RF energy sensing, jaw gap sensor (199), and jaw clamp sensor (125) in order to segment data acquisition based on when jaws (182, 184) are clamped and not clamped, suitably analyze tissue located between jaws (182, 184) while clamped, notify a user of suitable determinations, and/or log data for further use.

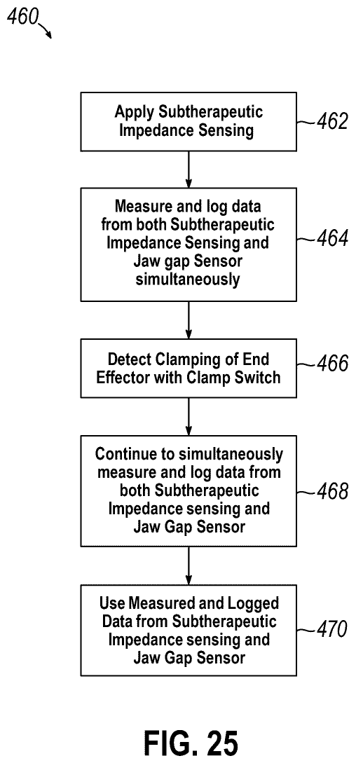

FIG. 25 shows an illustrative method (460) of using instrument (100′) in conjunction with subtherapeutic RF energy sensing, jaw gap sensor (199), and jaw clamp sensor (125) during illustrative use in accordance with the description herein. First, system (98) may apply subtherapeutic impedance sensing to jaws (182, 184) (i.e., frequency sweeps). Therefore, control unit (102) (or other suitable electrical components) may receive data (e.g., impedance magnitude and phase angles for various frequencies), where such data may be utilized in accordance with the description herein. As such, system (98) may measure and/or log (464) data from both subtherapeutic impedance sensing and jaw gap sensor (199) simultaneously. Such data may be utilized for any suitable purpose as would be apparent to one skilled in the art in view of the teachings herein. For example, the known jaw gap provided by data from jaw gap sensor (199) may be utilized to normalize or otherwise filter the data obtained from subtherapeutic impedance sensing.

Next, when a user actuates jaws (182, 184) into the clamped position, control unit (102) (or other suitable electrical components) may detect clamping of end effector (180′) via clamp switch (125). After clamp switch (125) detects jaws (182, 184) are fully clamped, control unit (102) may continue (468) to measure and log (468) data from both subtherapeutic impedance sensing and jaw gap sensor (199) simultaneously. However, since system (98) is now aware jaws (182, 184) are fully clamped, system (98) may segment data collected from both jaw gap sensor (199) and subtherapeutic impedance sensing between the time frame prior to clamping and a time frame after clamping is achieved, but prior to therapeutic energy delivery. Further, system (98) may then use (470) the measured and/or logged data from subtherapeutic impedance sensing and jaw gap sensor (199).

Identifying when clamping occurs may allow system (98) to identify the time frame between clamping and sealing in order to analyze impedance signals for tissue identification in accordance with the description herein. For example, as mentioned above, impedance measurements of grasped tissue follow a general trend after clamping occurs. If the impedance magnitudes gradually decrease along a smooth profile with a first known value range (as shown in FIG. 19) after clamping is identified, system (98) may determine a suitable skeletonized vessel is being grasped. However, if the measured impedance magnitudes include measurements and sudden variations (i.e., spikes) within a second known value range that is different than the first known value range, system (98) may determine that an undesirable bundle of vessels has been grasped. As another example, system (98) may also monitor the subtherapeutic impedance sensing data and the jaw gap data in order to provide additional analysis of tissue grasped in the time frame between clamping and sealing.

Therefore, having the ability to determine when jaws (182, 184) are initially clamped may allow system (98) to provide a more accurate analysis of data collected from subtherapeutic sensing and jaw gap sensor (199).

In the current method (460), subtherapeutic sensing and jaw gap measurements occur prior to, and after clamping is detected. It should be understood that this is merely optional. In some instances, measurements may initiate in response to jaw clamping being detected. In some instances, measurements may initiate immediately prior to jaw clamping being det3etged (via a signal from jaw gap sensor (199) or any other suitable structure.

### IV. ILLUSTRATIVE ARTICULATION TRACKING DEVICE FOR INSTRUMENT

As mentioned above, articulation assembly (110) is configured to deflect end effector (180) from the longitudinal axis (LA) defined by shaft assembly (140). As also mentioned above, rotation of articulation control (132) relative to body (122) will bend articulation assembly (110) in order to drive deflection of end effector (180) from the longitudinal axis (LA) defined by shaft assembly (140). In some instances, it may be desirable for system (98) to know and communicate the articulation angle which end effector (180) is deflected from the longitudinal axis (LA) defined by shaft assembly (140).

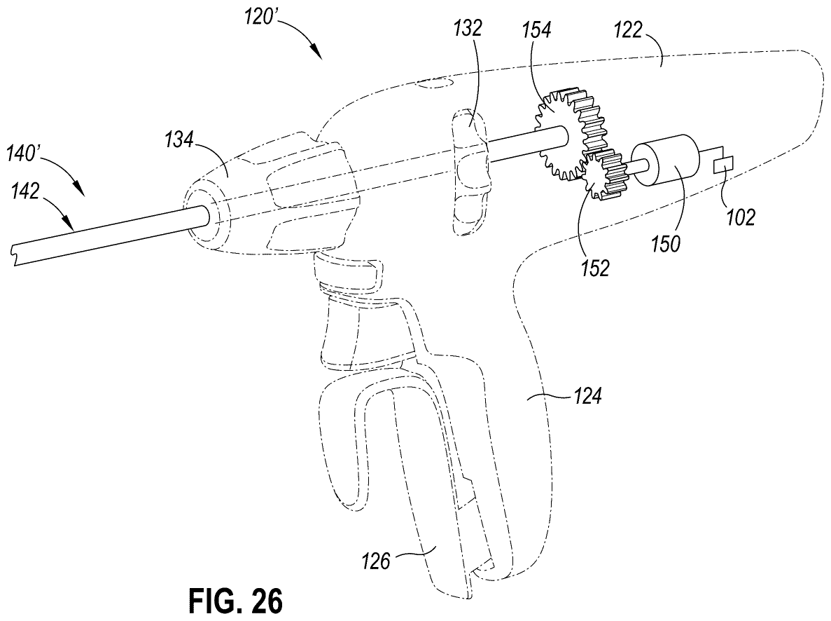

FIG. 26 shows an alternative handle assembly (120′) that may be readily incorporated into instrument (100, 100′) in replacement of handle assembly (120) described above. Therefore, handle assembly (120′) may be substantially similar to handle assembly (120) described above, except with the differences elaborated herein. In particular, handle assembly (120′) includes a rotary potentiometer (150) in electrical communication with control unit (102), while a suitable gear (154) is attached to articulation control (132). Gear (154) is attached is articulation control (132) such that rotation of articulation control (132) also rotates gear (154). Gear (154) meshes with intermediary gear (152) such that rotation of articulation control (132) also rotates intermediary gear (152). Rotary potentiometer (150) is suitably attached to intermediary gear (152) such that rotary potentiometer (150) may measure the rotational displacement of intermediary ger (152) and communicate that rotary displacement to control unit (102). Therefore, rotary potentiometer (150) is configured to measure the rotational displacement of articulation control (132). Therefore, control unit (102) may utilize data obtained from rotary potentiometer (150) in order to calculate the articulation angle which end effector (180) is deflected from the longitudinal axis (LA) defined by shaft assembly (140).

### V. ILLUSTRATIVE COMBINATIONS

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical instrument, comprising: (a) an end effector, the end effector comprising: (i) a first jaw, (ii) a second jaw, wherein the first jaw and the second jaw are configured to move relative to each other between an open position and a clamped position in order to grasp tissue between the first jaw and the second jaw in the clamped position, wherein the end effector is configured to apply a therapeutic energy to the grasped tissue in the clamped position to thereby seal the grasped tissue, (iii) a knife configured actuate from a pre-fired position toward a fired position to sever the grasped tissue, and (iv) an electrode associated with the first jaw, wherein the electrode is configured to engage the grasped tissue while the first jaw and the second jaw are in the clamped position, wherein the electrode is configured to emit non-therapeutic energy; and (b) a controller configured to measure and analyze an impedance phase angle and an impedance magnitude in response to the emitted non-therapeutic energy, wherein the controller is configured to detect a notable event at least partially based on the impedance phase angle or the impedance magnitude, wherein the notable event comprises at least one of the following: (i) the first jaw and the second jaw reaching the clamped position, (ii) the first jaw or the second jaw transitioning away from the clamped position toward the open position, or (iii) the knife actuating from the pre-fired position toward the fired position.

### Example 2

The surgical instrument of Example 1, wherein the controller is configured to generate a signal to display the notable event once detected.

### Example 3

The surgical instrument of any one or more of Examples 1-2, wherein the controller is configured to log the notable event once detected.

### Example 4

The surgical instrument of any one or more of Examples 1-3, wherein the controller is configured to, once the end effector ceases application of therapeutic energy, measure and analyze the impedance phase angle and generate a knife firing signal at least partially in response to the measured impedance phase angle.

### Example 5

The surgical instrument of Example 4, wherein the knife firing signal is configured to generate a visual display.

### Example 6

The surgical instrument of any one or more of Examples 1-5, wherein the controller is configured to segment data based on detection of the notable event.

### Example 7

The surgical instrument of any one or more of Examples 1-6, wherein the controller is configured to approximate a jaw gap between the first jaw and the second jaw in response to the impedance phase angle and the impedance magnitude.

### Example 8

The surgical instrument of Example 7, wherein the controller is configured to log the approximated jaw gap.

### Example 9

The surgical instrument of any one or more of Examples 1-9, further comprising a second electrode associated with the second jaw, wherein the electrode and the second electrode are both configured to engage tissue when the first jaw and the second jaw are in the clamped position.

### Example 10

The surgical instrument of Example 9, wherein the electrode and the second electrode are configured to apply therapeutic energy to the grasped tissue to thereby seal the grasped tissue.

### Example 11

A surgical instrument, comprising: (a) an end effector, the end effector comprising: (i) a first jaw, (ii) a second jaw, wherein the first jaw and the second jaw are configured to move relative to each other between an open position and a clamped position in order to grasp tissue between the first jaw and the second jaw in the clamped position, wherein the end effector is configured to apply a therapeutic energy to the grasped tissue in the clamped position to thereby seal the grasped tissue, and (iii) an electrode associated with the first jaw, wherein the electrode is configured to engage the grasped tissue while the first jaw and the second jaw are in the clamped position, wherein the electrode is configured to emit non-therapeutic energy; (b) a jaw gap sensor configured to measure a jaw gap between the first jaw and the second jaw, wherein the jaw gap sensor is configured to generate a jaw gap signal in response to the measured jaw gap; and (c) a controller in communication with the jaw gap sensor, wherein the controller is configured to measure an impedance value in response to the emitted non-therapeutic energy, wherein the controller is configured to utilize the impedance value and the jaw gap signal in order to identify a tissue property of the grasped tissue or alter a therapeutic energy delivery algorithm utilized by the end effector to apply the therapeutic energy to the grasped tissue.

### Example 12

The surgical instrument of Example 11, wherein the non-therapeutic energy comprises a plurality of predetermined frequencies.

### Example 13

The surgical instrument of Example 12, wherein the impedance value comprises a plurality of impedance magnitudes, each associated with a corresponding frequency of the plurality of predetermined frequencies.

### Example 14

The surgical instrument of any one or more of Examples 12-13, wherein the impedance value comprises a plurality of impedance phase angles, each associated with a corresponding frequency of the plurality of predetermined frequencies.

### Example 15

The surgical instrument of any one or more of Examples 11-14, wherein controller is configured to utilize the jaw gap signal and the measured impedance value to calculate a seal quality of sealed the grasped tissue.

### Example 16

A surgical instrument, comprising: (a) an end effector, the end effector comprising: (i) a first jaw, (ii) a second jaw, wherein the first jaw and the second jaw are configured to move relative to each other between an open position and a clamped position in order to grasp tissue between the first jaw and the second jaw in the clamped position, wherein the end effector is configured to apply a therapeutic energy to the grasped tissue in the clamped position to thereby seal the grasped tissue, and (iii) an electrode associated with the first jaw, wherein the electrode is configured to engage the grasped tissue while the first jaw and the second jaw are in the clamped position, wherein the electrode is configured to emit non-therapeutic energy; (b) a jaw gap sensor configured to measure a jaw gap between the first jaw and the second jaw, wherein the jaw gap sensor is configured to generate a jaw gap signal in response to the measured jaw gap; (c) a jaw clamp sensor configured to detect when the first jaw and the second jaw are in the clamped position and generate a jaw clamp signal; and (c) a controller in communication with both the jaw gap sensor and the jaw clamp sensor, wherein the controller is configured to measure an impedance value in response to the emitted non-therapeutic energy, wherein the controller is configured to utilize the impedance value, the jaw gap signal, and the jaw clamp signal in order to identify a tissue property of the grasped tissue or alter a therapeutic energy delivery algorithm utilized by the end effector to apply the therapeutic energy to the grasped tissue.

### Example 17

The surgical instrument of Example 16, wherein controller is configured to utilize the jaw gap signal, after initially receiving the jaw clamp signal, to infer a tissue-strain rate.

### Example 18

The surgical instrument of Example 17, wherein the controller is configured to utilize the tissue-strain rate to determine a tissue type of the grasped tissue.

### Example 19

The surgical instrument of any one or more of Examples 16-18, wherein the controller is configured to segment data after initially receiving the jaw clamp signal.

### Example 20

The surgical instrument of any one or more of Examples 16-19, further comprising a handle, wherein the jaw clamp sensor is attached to the handle.

### VI. MISCELLANEOUS

Any one or more of the teaching, expressions, embodiments, examples, etc. described herein may be combined with any one or more of the teachings, expressions, embodiments, examples, etc. described in U.S. Pat. App. No. [Atty. Ref. No. END9574USNP1], entitled “Electrosurgical Instrument with Jaw Status Monitoring and Method of Adjusting Energy Activation,” filed on even date herewith; U.S. Pat. App. No. [Atty. Ref. No. END9575USNP1], entitled “Electrosurgical Instrument and Method of Monitoring Clamp Position to Adjust Energy Application,” filed on even date herewith; U.S. Pat. App. No. [Atty. Ref. No. END9576USNP1], entitled “Electrosurgical Instrument and Method of Detecting Tissue Accumulation on End Effector,” filed on even date herewith; U.S. Pat. App. No. [Atty. Ref. No. END9577USNP1], entitled “Electrosurgical Instrument and Method of Applying Energy,” filed on even date herewith; and/or U.S. Pat. App. No. [Atty. Ref. No. END9609USNP1], entitled “Electrosurgical Instrument and Method of Frequency Monitoring for Sealing Tissue,” filed on even date herewith. The disclosure of each of these applications is incorporated by reference herein.

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those of ordinary skill in the art.

While the examples herein are described mainly in the context of electrosurgical instruments, it should be understood that various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, California. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein, in its entirety.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical instrument, comprising:
(a) an end effector, the end effector comprising:
(i) a first jaw,
(ii) a second jaw, wherein the first jaw and the second jaw are configured to move relative to each other between an open position and a clamped position in order to grasp tissue between the first jaw and the second jaw in the clamped position, wherein the end effector is configured to apply a therapeutic energy to the grasped tissue in the clamped position to thereby seal the grasped tissue,
(iii) a knife configured actuate from a pre-fired position toward a fired position to sever the grasped tissue, and
(iv) an electrode associated with the first jaw, wherein the electrode is configured to engage the grasped tissue while the first jaw and the second jaw are in the clamped position, wherein the electrode is configured to emit non-therapeutic energy; and
(b) a controller configured to measure and analyze an impedance phase angle and an impedance magnitude in response to the emitted non-therapeutic energy, wherein the controller is configured to detect a notable event at least partially based on the impedance phase angle or the impedance magnitude, wherein the notable event comprises at least one of the following:
(i) the first jaw and the second jaw reaching the clamped position,
(ii) the first jaw or the second jaw transitioning away from the clamped position toward the open position, or
(iii) the knife actuating from the pre-fired position toward the fired position.

2. The surgical instrument of claim 1, wherein the controller is configured to generate a signal to display the notable event once detected.

3. The surgical instrument of claim 1, wherein the controller is configured to log the notable event once detected.

4. The surgical instrument of claim 1, wherein the controller is configured to, once the end effector ceases application of therapeutic energy, measure and analyze the impedance phase angle and generate a knife firing signal at least partially in response to the measured impedance phase angle.

5. The surgical instrument of claim 4, wherein the knife firing signal is configured to generate a visual display.

6. The surgical instrument of claim 1, wherein the controller is configured to segment data based on detection of the notable event.

7. The surgical instrument of claim 1, wherein the controller is configured to approximate a jaw gap between the first jaw and the second jaw in response to the impedance phase angle and the impedance magnitude.

8. The surgical instrument of claim 7, wherein the controller is configured to log the approximated jaw gap.

9. The surgical instrument of claim 1, further comprising a second electrode associated with the second jaw, wherein the electrode and the second electrode are both configured to engage tissue when the first jaw and the second jaw are in the clamped position.

10. The surgical instrument of claim 9, wherein the electrode and the second electrode are configured to apply therapeutic energy to the grasped tissue to thereby seal the grasped tissue.

11. A surgical instrument, comprising:
(a) an end effector, the end effector comprising:
(i) a first jaw,
(ii) a second jaw, wherein the first jaw and the second jaw are configured to move relative to each other between an open position and a clamped position in order to grasp tissue between the first jaw and the second jaw in the clamped position, wherein the end effector is configured to apply a therapeutic energy to the grasped tissue in the clamped position to thereby seal the grasped tissue, and
(iii) an electrode associated with the first jaw, wherein the electrode is configured to engage the grasped tissue while the first jaw and the second jaw are in the clamped position, wherein the electrode is configured to emit non-therapeutic energy;
(b) a jaw gap sensor configured to measure a jaw gap between the first jaw and the second jaw, wherein the jaw gap sensor is configured to generate a jaw gap signal in response to the measured jaw gap; and
(c) a controller in communication with the jaw gap sensor, wherein the controller is configured to measure an impedance value in response to the emitted non-therapeutic energy, wherein the controller is configured to utilize the impedance value and the jaw gap signal in order to identify a tissue property of the grasped tissue or alter a therapeutic energy delivery algorithm utilized by the end effector to apply the therapeutic energy to the grasped tissue.

12. The surgical instrument of claim 11, wherein the non-therapeutic energy comprises a plurality of predetermined frequencies.

13. The surgical instrument of claim 12, wherein the impedance value comprises a plurality of impedance magnitudes, each associated with a corresponding frequency of the plurality of predetermined frequencies.

14. The surgical instrument of claim 12, wherein the impedance value comprises a plurality of impedance phase angles, each associated with a corresponding frequency of the plurality of predetermined frequencies.

15. The surgical instrument of claim 11, wherein controller is configured to utilize the jaw gap signal and the measured impedance value to calculate a seal quality of sealed the grasped tissue.

16. A surgical instrument, comprising:
(a) an end effector, the end effector comprising:
(i) a first jaw,
(ii) a second jaw, wherein the first jaw and the second jaw are configured to move relative to each other between an open position and a clamped position in order to grasp tissue between the first jaw and the second jaw in the clamped position, wherein the end effector is configured to apply a therapeutic energy to the grasped tissue in the clamped position to thereby seal the grasped tissue, and
(iii) an electrode associated with the first jaw, wherein the electrode is configured to engage the grasped tissue while the first jaw and the second jaw are in the clamped position, wherein the electrode is configured to emit non-therapeutic energy;
(b) a jaw gap sensor configured to measure a jaw gap between the first jaw and the second jaw, wherein the jaw gap sensor is configured to generate a jaw gap signal in response to the measured jaw gap;
(c) a jaw clamp sensor configured to detect when the first jaw and the second jaw are in the clamped position and generate a jaw clamp signal; and
(c) a controller in communication with both the jaw gap sensor and the jaw clamp sensor, wherein the controller is configured to measure an impedance value in response to the emitted non-therapeutic energy, wherein the controller is configured to utilize the impedance value, the jaw gap signal, and the jaw clamp signal in order to identify a tissue property of the grasped tissue or alter a therapeutic energy delivery algorithm utilized by the end effector to apply the therapeutic energy to the grasped tissue.

17. The surgical instrument of claim 16, wherein controller is configured to utilize the jaw gap signal, after initially receiving the jaw clamp signal, to infer a tissue-strain rate.

18. The surgical instrument of claim 17, wherein the controller is configured to utilize the tissue-strain rate to determine a tissue type of the grasped tissue.

19. The surgical instrument of claim 16, wherein the controller is configured to segment data after initially receiving the jaw clamp signal.

20. The surgical instrument of claim 16, further comprising a handle, wherein the jaw clamp sensor is attached to the handle.
